(19) United States
(12) Patent Application Publication
(10) Pub. No.: US 2023/0203031 A1
(43) Pub. Date: Jun. 29, 2023

(54) COMPOUNDS USEFUL FOR INHIBITING CDK7

(71) Applicant: Eli Lilly and Company, Indianapolis, IN (US)

(72) Inventors: Maria Carmen FERNANDEZ FIGUEROA, Madrid (ES); Wenceslao LUMERAS AMADOR, Madrid (ES); Concepcion SANCHEZ-MARTINEZ, Madrid (ES)

(21) Appl. No.: 17/998,861

(22) PCT Filed: May 20, 2021
(86) PCT No.: PCT/US2021/033414

§ 371 Date: Nov. 15, 2022

(30) Foreign Application Priority Data
May 27, 2020 (EP) 20382446.1

(51) Int. Cl. C07D 471/04; A61P 35/00

(52) U.S. Cl. CPC C07D 471/04; A61P 35/00

(57) ABSTRACT

CDK7 inhibitors according to the formula (I): wherein X is —CH(OH)CH₃, —CHFCH₃, —CF₂CH₃, or —CF₃; Y is —CH═CH₂ or C²H═C²H₂; and Z is CH(CH₃)₂ or C²H(CH₃)(CH₂²H), pharmaceutically acceptable salts thereof, pharmaceutical compositions thereof, and methods for their use are provided.

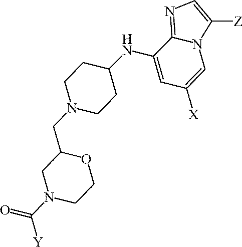

Cyclin-dependent kinases are a major class of kinases that are important in cancer cell proliferation and deregulated oncogenic transcription. CDK7 is a cyclin-dependent kinase that binds to cyclin H and MATI to form a trimeric cyclin-activating kinase that performs its function by phosphorylating other cyclin-activating kinases involved in cell-cycle control. These complexes control specific transitions between two subsequent phases in the cell cycle. CDK7 is implicated in both temporal control of the cell cycle and transcriptional activity. CDK7 is implicated in the transcriptional initiation process by phosphorylation of Rbp1 subunit of RNA Polymerase II. Uncontrolled cell proliferation and deregulated transcription is a cancer hallmark. Targeting CDK7 selectively may offer an advantage by simultaneously inhibiting active transcription and cell-cycle progression. Therefore, CDK7 is a promising target for the treatment of cancer, in particular aggressive and hard-to-treat cancers.

Some small molecule inhibitors against CDK7 have been reported in the literature (see, e.g., WO 2015/154022, WO 2016/142855, WO 2016/160617, WO 2016/193939, and WO 2017/044858). However, known CDK7 inhibitors may not be specific to CDK7 and have not yet been as useful as is needed to effectively treat cell proliferative disorders, such as cancer. Thus, there remains a need to provide new selective CDK7 inhibitors to treat cell proliferative disorders.

Compounds of the formula:

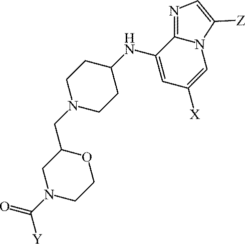

pharmaceutically acceptable salts thereof, or pharmaceutical compositions thereof, are provided herein. In this formula, X can be —CH(OH)CH₃, —CHFCH₃, —CF₂CH₃ or —CF₃; Y can be —CH═CH₂ or —C²H═C²H₂; and Z can be —CH(CH₃)₂ or —C²H(CH₃)(CH₂²H).

The compounds of this formula contain a chiral center providing an R-enantiomeric form shown and S-enantiomeric form as shown here:

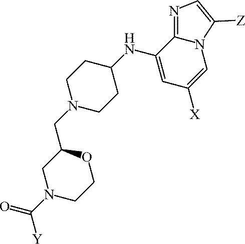

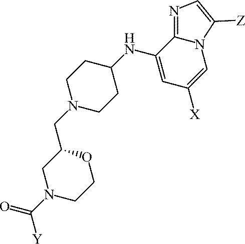

The R-enantiomer and S-enantiomer, pharmaceutically acceptable salts thereof, or pharmaceutical compositions thereof, in which X, Y, and Z are defined as above, are also provided herein.

Methods of using the compounds of this formula, pharmaceutically acceptable salts thereof, and pharmaceutical compositions thereof, to treat urothelial cancer, uterine cancer, colorectal cancer, breast cancer, lung cancer, ovarian cancer, gastric cancer, hepatobiliary cancer, pancreatic cancer, cervical cancers, prostate cancer, hematological cancers, sarcomas, skin cancers, or gliomas are also provided. The methods include administering a therapeutically effective amount of a compound of this formula, or a pharmaceutically acceptable salt thereof, to a patient in need. The methods can also include testing for the presence of at least one loss of function mutation in the ARID1A, KMT2C, KMT2D, or RBI gene in a biological sample from a patient and administering a therapeutically effective amount of a compound of this formula, or a pharmaceutically acceptable salt thereof, to the patient if the sample tests positive for the loss of function mutation. The methods can further or alternatively include administering a therapeutically effective amount of a compound of this formula, or a pharmaceutically acceptable salt thereof, to the patient provided that a biological sample from the patient contains at least one loss of function mutation in the ARID1A, KMT2C, KMT2D, or RBI gene. The methods can additionally or alternatively include administering a therapeutically effective amount of a compound of this formula, or a pharmaceutically acceptable salt thereof, to the patient provided that the patient is selected for treatment if a biological sample from the patient tests positive for at least one loss of function mutation in the ARID1A, KMT2C, KMT2D, or RBI gene.

Also provided herein, are the compounds of this formula, and pharmaceutically acceptable salts thereof, for use in therapy. Also provided herein, are the compounds of this formula, and pharmaceutically acceptable salts thereof, for use in the treatment of urothelial cancer, uterine cancer, colorectal cancer, breast cancer, lung cancer, ovarian cancer, gastric cancer, hepatobiliary cancer, pancreatic cancer, cervical cancers, prostate cancer, hematological cancers, sarcomas, skin cancers, or gliomas. For further example, the treatment can include performing an in vitro assay using a biological sample from the patient, determining the presence of at least one inactivating mutation in the ARID1A, KMT2C, KMT2D, and RBI genes, and administering a therapeutically effective amount of a compound of this formula, or pharmaceutically acceptable salts thereof, to the patient if at least one inactivating mutation in any of the genes is present.

The use of a compound of this formula, or pharmaceutically acceptable salts thereof, in the manufacture of a medicament for treating a urothelial cancer, uterine cancer, colorectal cancer, breast cancer, lung cancer, ovarian cancer, gastric cancer, hepatobiliary cancer, pancreatic cancer, cervical cancers, prostate cancer, hematological cancers, sarcomas, skin cancers, or gliomas is also provided. This use can include performing an in vitro assay using a biological sample from the patient, determining the presence of at least one inactivating mutation in the ARID1A, KMT2C, KMT2D, and RBI genes, and administering a therapeutically effective amount of a compound of this formula, including R- and S-enantiomeric forms, or pharmaceutically acceptable salts thereof, to the patient if at least one inactivating mutation in any of the genes is present.

### DESCRIPTION

Novel selective CDK7 inhibitor compounds are described herein. These new compounds could address the need for potent, effective treatment of cancer, especially cancer stemming from deregulated transcription. More specifically, these new compounds could address the need for potent, effective treatment of urothelial cancer, uterine cancer, colorectal cancer, breast cancer, lung cancer, ovarian cancer, gastric cancer, hepatobiliary cancer, pancreatic cancer, cervical cancers, prostate cancer, hematological cancers, sarcomas, skin cancers, and/or gliomas.

The compounds described herein are compounds of formula (I):

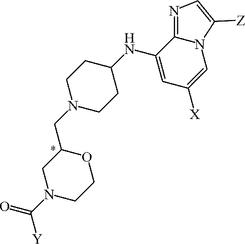

or pharmaceutically acceptable salts thereof. In formula (I), X is —CH(OH)CH₃,—CHFCH₃, —CF₂CH₃ or —CF₃; Y is —CH═CH₂ or —C²H═C²H₂ and Z is —CH(CH₃)₂ or —C²H(CH₃)(CH₂²H). Specific examples of formula (I) include compounds in which X is —CH(OH)CH₃, —CHFCH₃, or —CF₂CH₃; Y is —CH═CH₂; and Z is —CH(CH₃)₂. Further examples formula (I) include compounds in which X is —CF₃; Y is —CH═CH₂ or —C²H═C²H₂; and Z is —CH(CH₃)₂ or —C²H(CH₃)(CH₂²H). One of skill in the art will appreciate that compounds as described by formula (I), or pharmaceutically acceptable salts thereof, contain a chiral center, the position of which is indicated by an * above. One of skill in the art will also appreciate that the Cahn-Ingold-Prelog (R) or (S) designations for chiral centers will vary depending upon the substitution patterns around a chiral center. The chiral center in the compound of formula (I) provides an R-enantiomeric form shown by formula (II) and an S-enantiomeric from shown by formula (III):

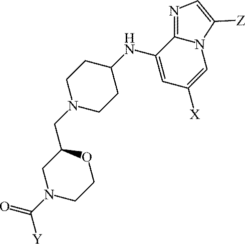

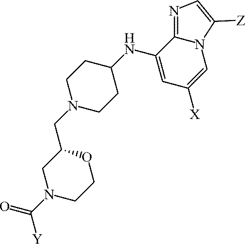

Compounds of formula (II) and formula (III) or pharmaceutically acceptable salts thereof, in which X, Y, and Z are defined as for formula (I), are also provided herein.

Specific enantiomers may be prepared beginning with chiral reagents or by stereoselective or stereo-specific synthetic techniques. Alternatively, single enantiomers may be isolated from mixtures of different chiral forms by standard chiral chromatographic or crystallization techniques at any convenient point in the synthesis of compounds of formula (I), formula (II), and formula (III). All individual enantiomers, as well as mixtures of the enantiomers of the compounds of formula (II) and formula (III) including racemates are intended to be included herein.

Specific examples of the compounds of formula (II) (including IUPAC nomenclature names) are shown here:

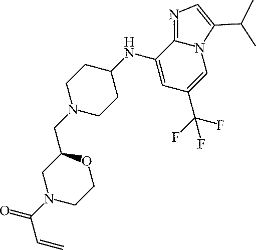 [SMILES: C=CC(=O)N1CCO[C@H](CN2CCC(Nc3cc(C(F)(F)F)cn4c(C(C)C)cnc34)CC2)C1]

1-[(2R)-2-[[4-[[3-isopropyl-6-(trifluoromethyl)imidazo[1,2-a]pyridin-8-yl]amino]-1-piperidyl]methyl]morpholin-4-yl]prop-2-en-1-one;

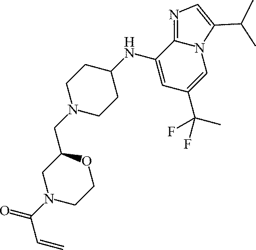 [SMILES: C=CC(=O)N1CCO[C@H](CN2CCC(Nc3cc(C(C)(F)F)cn4c(C(C)C)cnc34)CC2)C1]

1-[(2R)-2-[[4-[[6-(1,1-difluoroethyl)-3-isopropyl-imidazo[1,2-a]pyridin-8-yl]amino]-1-piperidyl]methyl]morpholin-4-yl]prop-2-en-1-one;

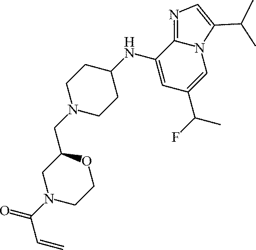 [SMILES: C=CC(=O)N1CCO[C@H](CN2CCC(Nc3cc(C(C)F)cn4c(C(C)C)cnc34)CC2)C1]

1-[(2R)-2-[[4-[[6-(1-fluoroethyl)-3-isopropyl-imidazo[1,2-a]pyridin-8-yl]amino]-1-piperidyl]methyl]morpholin-4-yl]prop-2-en-1-one;

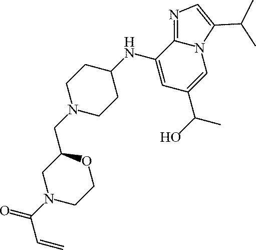 [SMILES: C=CC(=O)N1CCO[C@H](CN2CCC(Nc3cc(C(C)O)cn4c(C(C)C)cnc34)CC2)C1]

1-[(2R)-2-[[4-[[6-(1-hydroxyethyl)-3-isopropyl-imidazo[1,2-a]pyridin-8-yl]amino]-1-piperidyl]methyl]morpholin-4-yl]prop-2-en-1-one;

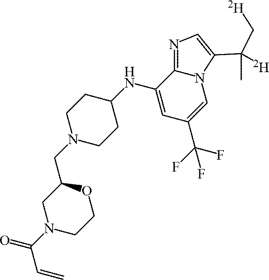 [SMILES: [2H]CC([2H])(C)c1cnc2c(NC3CCN(C[C@@H]4CN(C(=O)C=C)CCO4)CC3)cc(C(F)(F)F)cn12]

1-[(2R)-2-[[4-[[3-(1,2-dideuterio-1-methyl-ethyl)-6-(trifluoromethyl)imidazo[1,2-a]pyridin-8-yl]amino]-1-piperidyl]methyl]morpholin-4-yl]prop-2-en-1-one; and

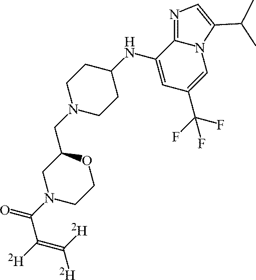 [SMILES: [2H]C([2H])=C([2H])C(=O)N1CCO[C@H](CN2CCC(Nc3cc(C(F)(F)F)cn4c(C(C)C)cnc34)CC2)C1]

2,3,3-trideuterio-1-[(2R)-2-[[4-[[3-isopropyl-6-(trifluoromethyl)imidazo[1,2-a]pyridin-8-yl]amino]-1-piperidyl]methyl]morpholin-4-yl]prop-2-en-1-one.

Specific examples of the compounds of formula (III) (including IUPAC nomenclature names) are shown here:

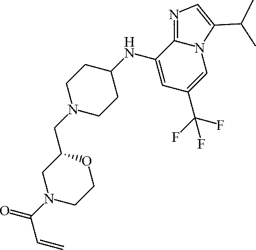 [SMILES: C=CC(=O)N1CCO[C@@H](CN2CCC(Nc3cc(C(F)(F)F)cn4c(C(C)C)cnc34)CC2)C1]

1-[(2S)-2-[[4-[[3-isopropyl-6-(trifluoromethyl)imidazo[1,2-a]pyridin-8-yl]amino]-1-piperidyl]methyl]morpholin-4-yl]prop-2-en-1-one;

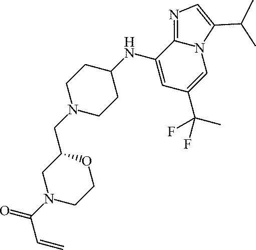 [SMILES: C=CC(=O)N1CCO[C@@H](CN2CCC(Nc3cc(C(C)(F)F)cn4c(C(C)C)cnc34)CC2)C1]

1-[(2S)-2-[[4-[[6-(1,1-difluoroethyl)-3-isopropyl-imidazo[1,2-a]pyridin-8-yl]amino]-1-piperidyl]methyl]morpholin-4-yl]prop-2-en-1-one;

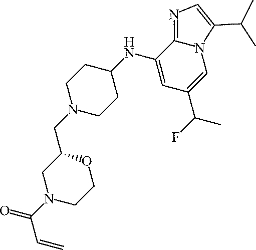 [SMILES: C=CC(=O)N1CCO[C@@H](CN2CCC(Nc3cc(C(C)F)cn4c(C(C)C)cnc34)CC2)C1]

1-[(2S)-2-[[4-[[6-(1-fluoroethyl)-3-isopropyl-imidazo[1,2-a]pyridin-8-yl]amino]-1-piperidyl]methyl]morpholin-4-yl]prop-2-en-1-one;

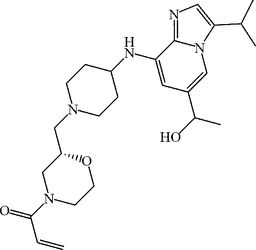 [SMILES: C=CC(=O)N1CCO[C@@H](CN2CCC(Nc3cc(C(C)O)cn4c(C(C)C)cnc34)CC2)C1]

1-[(2S)-2-[[4-[[6-(1-hydroxyethyl)-3-isopropyl-imidazo[1,2-a]pyridin-8-yl]amino]-1-piperidyl]methyl]morpholin-4-yl]prop-2-en-1-one;

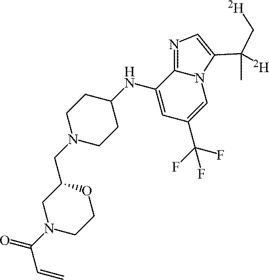 [SMILES: [2H]CC([2H])(C)c1cnc2c(NC3CCN(C[C@H]4CN(C(=O)C=C)CCO4)CC3)cc(C(F)(F)F)cn12]

1-[(2S)-2-[[4-[[3-(1,2-dideuterio-1-methyl-ethyl)-6-(trifluoromethyl)imidazo[1,2-a]pyridin-8-yl]amino]-1-piperidyl]methyl]morpholin-4-yl]prop-2-en-1-one; and

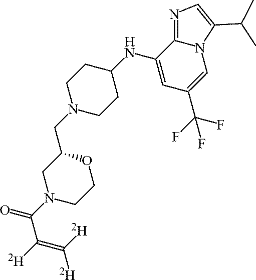 [SMILES: [2H]C([2H])=C([2H])C(=O)N1CCO[C@@H](CN2CCC(Nc3cc(C(F)(F)F)cn4c(C(C)C)cnc34)CC2)C1]

2,3,3-trideuterio-1-[(2S)-2-[[4-[[3-isopropyl-6-(trifluoromethyl)imidazo[1,2-a]pyridin-8-yl]amino]-1-piperidyl]methyl]morpholin-4-yl]prop-2-en-1-one.

Several deuterated molecules are specifically described herein, e.g., compounds of formula (I) where Y is —C²H═C²H₂ and where Z is —C²H(CH₃)(CH₂²H). Other deuterated molecules are possible and are considered to be disclosed herein where a hydrogen can be replaced by a deuterium in a disclosed molecule.

The compounds described herein may react to form pharmaceutically acceptable salts and pharmaceutically acceptable salts of the compounds of formula (I), formula (II), and formula (III) as well as the specific examples of the compounds of formula (I), formula (II), and formula (III) are intended to be included. Pharmaceutically acceptable salts and common methodology for preparing them are well known in the art (see, e.g., P. Stahl, et al. Handbook of Pharmaceutical Salts: Properties, Selection and Use, 2^{nd} Revised Edition (Wiley-VCH, 2011); S.M. Berge, et al., “Pharmaceutical Salts,” Journal of Pharmaceutical Sciences, Vol. 66, No. 1, January 1977). Specific examples of useful pharmaceutically acceptable salts include hydrochloride salts and sulfate salts, but this list is not intended to be exclusive.

The compounds described herein are generally effective over a wide dosage range. For example, dosages per day fall within the range of about 1 mg to about 2 g. It will be understood that the amount of the compound actually administered will be determined by a physician, in light of the relevant circumstances, including the condition to be treated, the chosen route of administration, the actual compound or compounds administered, the age, weight, and response of the individual patient, and the severity of the patient’s symptoms.

The compounds described herein can be formulated as pharmaceutical compositions that can be administered by a variety of routes. Such pharmaceutical compositions and processes for preparing the same are well known in the art (see, e.g., Remington: The Science and Practice of Pharmacy (A. Gennaro, et al., eds., 21st ed., Mack Publishing Co., 2005)). Specifically, the compounds of formula (I), formula (II), and formula (III) as described herein, or pharmaceutically acceptable salts thereof, can be combined with one or more pharmaceutically acceptable carriers, diluents, or excipients. More particularly, the compounds described herein by formula (I), formula (II), and formula (III) can be formulated as pharmaceutical compositions. Further, the compounds of formula (I), formula (II), and formula (III) as described herein, or pharmaceutically acceptable salts thereof, can be combined with one or more other therapeutic agents. For example, the compounds of formula (I), formula (II), and formula (III) as described herein, or pharmaceutically acceptable salts thereof, can be a component in a pharmaceutical composition for the treatment of cancer in combination with one or more pharmaceutically acceptable carriers, diluents, or excipients, and optionally with one or more additional therapeutic agents. Pharmaceutical compositions containing the compounds of formula (I), formula (II), and formula (III) as described herein, or pharmaceutically acceptable salts thereof, can be used in the methods described herein.

The term “treating” (or “treat” or “treatment”) as used herein refers to restraining, slowing, stopping, or reversing the progression or severity of an existing symptom, condition or disorder.

As used herein, the terms “cancer” and “cancerous” refer to or describe the physiological condition in patients that is typically characterized by unregulated cell proliferation. Included in this definition are benign and malignant cancers. By “early stage cancer” or “early stage tumor” is meant a cancer that is not advanced or metastatic or is classified as a Stage 0, I, or II cancer. Examples of cancer include, but are not limited to, urothelial cancer, uterine cancer, colorectal cancer, breast cancer, lung cancer, ovarian cancer, gastric cancer, hepatobiliary cancer, pancreatic cancer, cervical cancers, prostate cancer, hematological cancers, sarcomas, skin cancers, or gliomas.

Methods for the treatment of cancer, in particular for the treatment of cancer with deregulated transcription using the compounds of formula (I), formula (II), or formula (III) as described herein are provided. One such method includes administering a therapeutically effective amount of a compound of formula (I), formula (II), or formula (III) as described herein to a patient in need thereof. The types of cancer that can be treated using the compositions described herein include urothelial cancer, uterine cancer, colorectal cancer, breast cancer, lung cancer, ovarian cancer, gastric cancer, hepatobiliary cancer, pancreatic cancer, cervical cancers, prostate cancer, hematological cancers, sarcomas, skin cancers, or gliomas. More specifically, the types of cancer can be colorectal cancer, breast cancer, lung cancer, ovarian cancer, or gastric cancer. Specifically, the cancer can be breast cancer. These types of cancers can be associated with a loss of function mutation in the ARID1A, KMT2C, KMT2D, or RBI genes. Thus, a loss of function mutation in an ARID1A, KMT2C, KMT2D, or RBI gene can be an indication treatment is needed. A loss of function mutation in one or more of the ARID1A, KMT2C, KMT2D, or RBI genes can be an indication that treatment with one or more of the methods described herein could be useful.

Another method of treating urothelial cancer, uterine cancer, colorectal cancer, breast cancer, lung cancer, ovarian cancer, gastric cancer, hepatobiliary cancer, pancreatic cancer, cervical cancers, prostate cancer, hematological cancers, sarcomas, skin cancers, or gliomas in a patient, includes testing for the presence of at least one loss of function mutation in an ARID1A, KMT2C, KMT2D, or RBI gene in a biological sample from a patient and administering a therapeutically effective amount of a compound of formula (I), formula (II), or formula (III) as described herein, or a pharmaceutically acceptable salt thereof, to the patient if the biological sample tests positive for at least one loss of function mutation in any of an ARID1A, KMT2C, KMT2D, or RBI gene.

A further method of treating urothelial cancer, uterine cancer, colorectal cancer, breast cancer, lung cancer, ovarian cancer, gastric cancer, hepatobiliary cancer, pancreatic cancer, cervical cancers, prostate cancer, hematological cancers, sarcomas, skin cancers, or gliomas in a patient, includes administering a therapeutically effective amount of a compound of formula (I), formula (II), or formula (III) as described herein, or a pharmaceutically acceptable salt thereof, to a patient provided that a biological sample from the patient contains at least one loss of function mutation in an ARID1A, KMT2C, KMT2D, or RBI gene.

An additional method of treating urothelial cancer, uterine cancer, colorectal cancer, breast cancer, lung cancer, ovarian cancer, gastric cancer, hepatobiliary cancer, pancreatic cancer, cervical cancers, prostate cancer, hematological cancers, sarcomas, skin cancers, or gliomas in a patient, includes administering a therapeutically effective amount of a compound of formula (I), formula (II), or formula (III) as described herein, or a pharmaceutically acceptable salt thereof, to a patient provided that the patient is selected for treatment if a biological sample from the patient tests positive for at least one loss of function mutation in an ARID1A, KMT2C, KMT2D, or RBI gene.

In the methods described herein, a biological sample can be a tumor sample. When a biological sample is obtained, the sample can be analyzed using methods known to those of skill in the art such as genomic/DNA sequencing. In the methods, a sample can be obtained from a patient prior to the first administration of a compound of formula (I), formula (II), or formula (III) as described herein, or a pharmaceutically acceptable salt thereof.

The compounds of formula (I), formula (II), and formula (III) as described herein, or pharmaceutically acceptable salts thereof, are also for use in therapy and in particular, for the treatment of cancer with deregulated transcription. As noted herein, cancers with deregulated transcription include urothelial cancer, uterine cancer, colorectal cancer, breast cancer, lung cancer, ovarian cancer, gastric cancer, hepatobiliary cancer, pancreatic cancer, cervical cancers, prostate cancer, hematological cancers, sarcomas, skin cancers, or gliomas. More specifically, the types of cancer can be colorectal cancer, breast cancer, lung cancer, ovarian cancer, or gastric cancer. Specifically, the cancer can be breast cancer. The compound of formula (I), formula (II) or formula (III), or a pharmaceutically acceptable salt thereof, may be administered to a patient having at least one inactivating mutation in the ARID1A, KMT2C, KMT2D, or RBI genes as determined by performing an in-vitro assay using a biological sample from the patient. The biological sample can be a tumor sample and, the tumor sample can be analyzed using methods known to those of skill in the art such as genomic/DNA sequencing. Additionally, the sample can be obtained from the patient prior to the first administration of the compound of formulas (I), (II), or (III) as described herein, or pharmaceutically acceptable salts thereof. Use of the compound of formula (I), formula (II), and formula (III) as described herein, or pharmaceutically acceptable salts thereof in a therapy can be based upon a patient being selected for treatment by having at least one inactivating mutation in an ARID1A, KMT2C, KMT2D, or RBI gene. When used in a therapy a compound of formula (I), formula (II), or formula (III) as described herein, or pharmaceutically acceptable salts thereof, may be administered to the patient at a dose of about 1 mg to 2 g.

A compound of formula (I), formula (II), or formula (III) as described herein, or pharmaceutically acceptable salts thereof, can be used in the manufacture of a medicament for the treatment of cancer. Cancers that can be treated using a medicament as described herein include urothelial cancer, uterine cancer, colorectal cancer, breast cancer, lung cancer, ovarian cancer, gastric cancer, hepatobiliary cancer, pancreatic cancer, cervical cancers, prostate cancer, hematological cancers, sarcomas, skin cancers, or gliomas. More specifically, the types of cancer can be colorectal cancer, breast cancer, lung cancer, ovarian cancer, or gastric cancer. Specifically, the cancer can be breast cancer. Use of a compound of formula (I), formula (II), or formula (III) as described herein, or pharmaceutically acceptable salts thereof, in the manufacture of a medicament can also include a step of performing an in vitro assay using a biological sample from a patient, determining the presence of at least one inactivating mutation in an ARID1A, KMT2C, KMT2D, or RBI gene, and administering a therapeutically effective amount of the compound of formula (I), formula (II), or formula (III) as described herein, or pharmaceutically acceptable salts thereof, to the patient if at least one inactivating mutation in any of the genes is present. In these uses, the biological sample can be a tumor sample and the tumor sample can be analyzed using methods known to those of skill in the art such as genomic/DNA sequencing. Additionally, in these uses the sample can be obtained from the patient prior to the first administration of the compound of formula (I), formula (II), and formula (III) as described herein, or pharmaceutically acceptable salts thereof. In these uses of the compound of formula (I), formula (II), and formula (III) as described herein, or pharmaceutically acceptable salts thereof in a therapy can be based upon a patient being selected for treatment by having at least one inactivating mutation in an ARID1A, KMT2C, KMT2D, or RBI gene. Also, in these uses a compound of formula (I), formula (II), or formula (III) as described herein, or pharmaceutically acceptable salts thereof, may be administered to the patient at a dose of about 1 mg to 2 g.

The compounds of formula (I), formula (II), and formula (III), or pharmaceutically acceptable salts thereof, may be prepared by a variety of procedures known in the art, as well as the Preparations and Examples below. The specific synthetic steps for each of the routes described may be combined in different ways, or in conjunction with steps from different schemes, to prepare compounds of formula (I), formula (II), and formula (III), or pharmaceutically acceptable salts thereof. The products of each step in the schemes below can be recovered by conventional methods well known in the art, including extraction, evaporation, precipitation, chromatography, filtration, trituration, and crystallization. The reagents and starting materials are readily available to one of ordinary skill in the art.

Individual isomers and enantiomers may be separated or resolved by one of ordinary skill in the art at any convenient point in the synthesis of the compounds described herein, for example, by methods such as selective crystallization techniques or chiral chromatography (see, for example, J. Jacques, et al., “Enantiomers, Racemates, and Resolutions″, John Wiley and Sons, Inc., 1981, and E.L. Eliel and S.H. Wilen,” Stereochemistry of Organic Compounds″, Wiley-Interscience, 1994).

Intermediates and processes useful for the synthesis of the compounds described by formula (I), formula (II), and formula (III) are intended to be included in this description.

Additionally, certain intermediates described herein may contain one or more protecting groups. The variable protecting group may be the same or different in each occurrence depending on the particular reaction conditions and the particular transformations to be performed. The protection and deprotection conditions are well known to the skilled artisan and are described in the literature (See for example “Greene’s Protective Groups in Organic Synthesis”, Fourth Edition, by Peter G.M. Wuts and Theodora W. Greene, John Wiley and Sons, Inc. 2007).

The following preparations and examples are presented to illustrate the methods and compounds described herein.

### Preparations and Examples

Certain abbreviations are defined as follows: “¹H NMR” refers to ¹H-nuclear magnetic resonance; “eq” refers to equivalent; “THF” refers to tetrahydrofuran; “DCM” refers to dichloromethane; “NCS” refers to N-chlorosuccinimide; “NIS” refers to N-iodosuccinimide; “IPA” refers to isopropyl alcohol; “ACN” refers to acetonitrile; “DIPEA” refers to N,N-diisopropylethylamine; “DMSO” refers to dimethyl sulfoxide; “EtOH” refers to ethanol; “MTBE” refers to methyl tert-butyl ether; “TEA” refers to triethylamine; “2-MeTHF” refers to 2-methyltetrahydrofuran; “MeOH” refers to methanol; “UV” refers to ultraviolet; “RP-LC/MS” refers to reverse phase liquid chromatography mass spectrometry; “ES/MS” refers to electrospray mass spectrometry; “DMEA” refers to dimethylethanolamine; “DMAP” refers to dimethylaminopyridine; “EtOAc” refers to ethyl acetate; “DMF” refers to N,N-dimethylformamide; “TFA” refers to trifluoroacetic acid; “SCX” refers to strong cation exchange; “e.e.” refers to enantiomeric excess; “min” refers to minutes; “h” refers to hours; “ATP” refers to adenosine triphosphate; “DTT” refers to dithiothreitol; “HEPES” refers to (4-(2-hydroxyethyl)-1-piperazineethanesulfonic acid); “EDTA” refers to Ethylenediaminetetraacetic acid; “ATCC” refers to American Type Culture Collection; “RT” refers to room temperature; “Rt” refers to retention time; “PBS” refers to phosphate-buffered saline; “BSA” refers to bovine serum albumin; “FBS” refers to fetal bovine serum; “RNAase” refers to ribonuclease; and “His” refers to histidine.

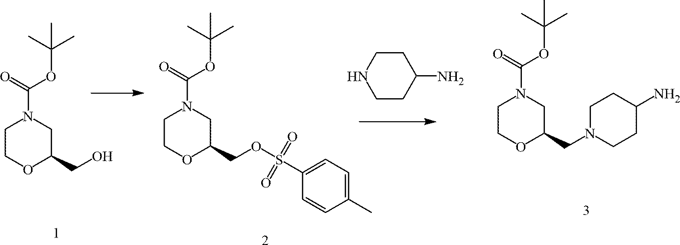

Scheme 1 depicts the synthesis of compound 3. Commercially available chiral hydroxymethyl morpholine 1 may be converted to p-toluene sulfonate 2 using the appropriate base. Sulfonate 2 may then be displaced via nucleophilic substitution with commercially available 4-aminopiperidine to provide the chiral N-protected morpholino piperidine primary amine 3.

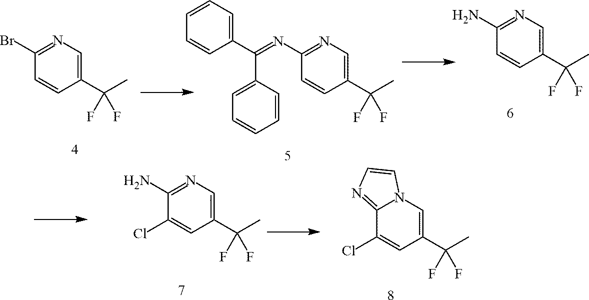

Scheme 2 depicts the synthesis of compound 8. Pyridylmethanimine 5 may be synthesized by treating commercially available difluoroethylpyridine 4 with diphenylmethanimine under metal catalyzed (e.g. Pd) coupling conditions well known in the art. Imine 5 may be deprotected under acidic conditions to provide 2-aminopyridine 6. Regioselective addition of chlorine may be accomplished by employing a suitable chlorinating agent to furnish chloropyridine 7. Synthesis of imidazopyridine 8 from 2-aminopyridine 7 may be carried out under a variety of conditions known to the skilled artisan including but not limited to cyclocondensations, rearrangements, and oxidative cyclizations.

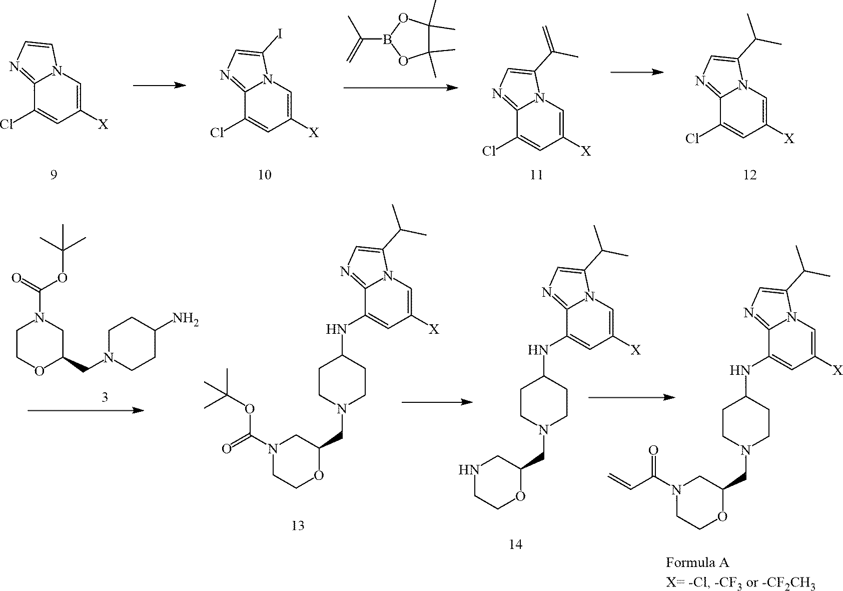

Scheme 3 depicts the synthesis of compounds of Formula A. Iodination of imidazopyridine 9 may be achieved with treatment of the proper iodine containing reagent (e.g. NIS, I₂) to furnish 3-iodoimidazopyridine 10. Subsequent coupling of 3-iodoimidazopyridine 10 may be achieved under a variety of conditions well known to the skilled artisan including metal catalyzed (e.g. Pd, Ni) reactions to provide isopropenyl imidazopyridine 11. Isopropyl imidazopyridine 12 may be synthesized from isopropenyl imidazopyridine 11 using reductive conditions including, but not limited to, Pd/C under a H₂ gas atmosphere. The aryl chloride of compound 12 may be displaced with 4-aminopiperidine 3 to provide aminoimidazopyridine 13. Deprotection of N-protected morpholine 13 may be achieved by treatment with the appropriate strong acid to provide secondary amine 14. The acrylamide Formula A may be formed by treatment of secondary amine 14 with base and the appropriate acid chloride.

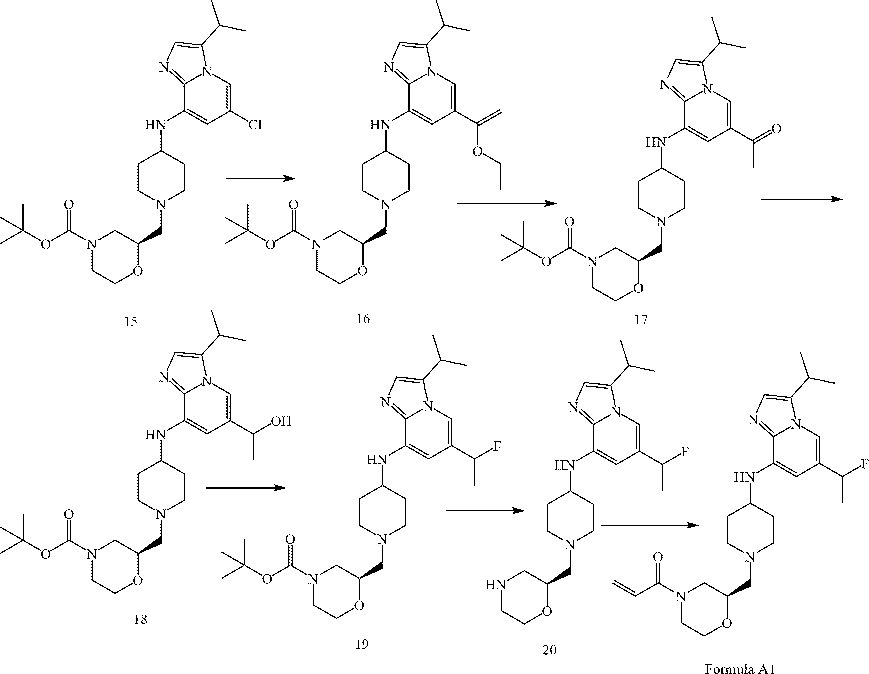

Scheme 4 depicts the synthesis of the compounds of Formula A1. Heteroaryl enol ether 16 may be synthesized from heteroaryl chloride 15 using the appropriate tin reagent and metal catalysis. Treatment of enol ether 16 with the appropriate aqueous strong acid results in heteroaryl ketone 17. Subsequent reduction to secondary alcohol 18 may be affected using an array of reducing agents, such as with a metal hydride, borohydride salt, or diborane in a polar aprotic solvent. Secondary alcohol 18 may be converted to benzyl fluoride 19 using the appropriate fluorinating reagent such as DAST, Deoxofluor, or XtalFluor. Formula A1 may then be prepared essentially as described in Scheme 3.

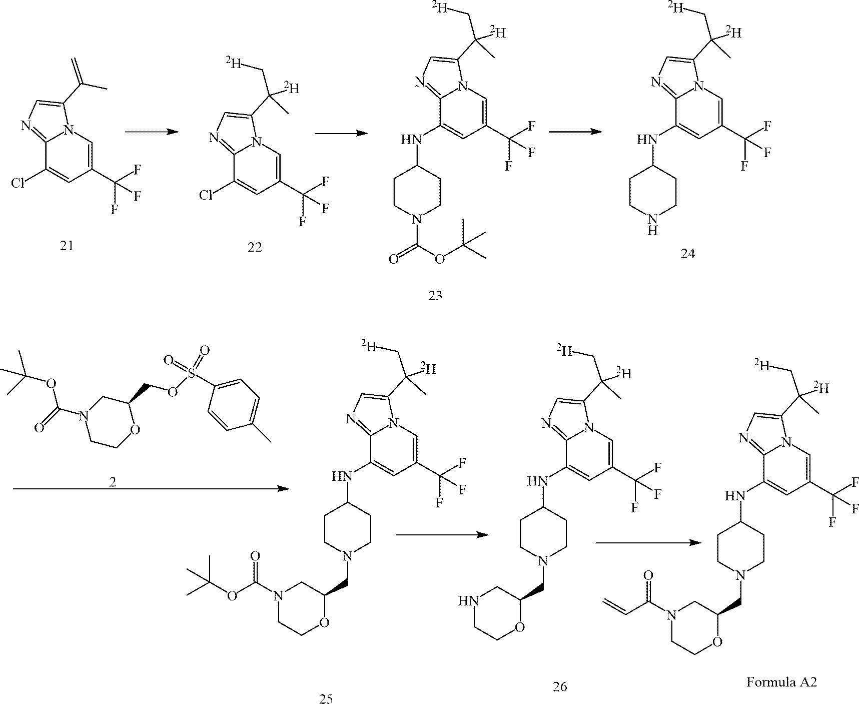

Scheme 5 depicts the synthesis of the compounds of Formula A2. Deuterated isopropyl imidazopyridine 22 may be prepared from isopropenyl imidazopyridine 21 using transition metal catalysis under a pressurized atmosphere of deuterium at elevated temperature. Heteroaryl chloride 22 may be substituted by nucleophilic displacement with N-protected 4-aminopiperidine essentially as described in Scheme 3 and deprotected to secondary amine using the appropriate strong acid. Piperidine 24 may be substituted with N-protected morpholinosulfonate 2 essentially as described in Scheme 1 and carried through to Formula A2 essentially as described in Scheme 3.

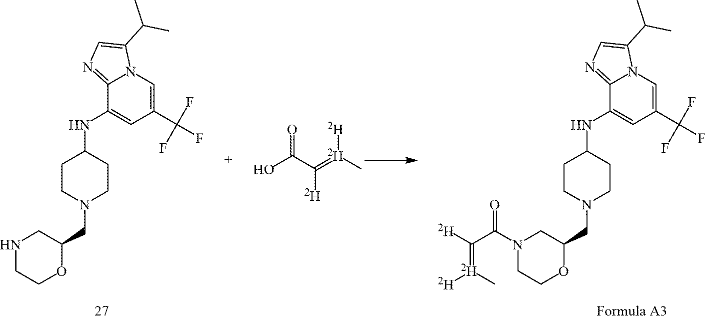

Scheme 6 depicts the synthesis of Formula A3 which may be made essentially as described in Scheme 3.

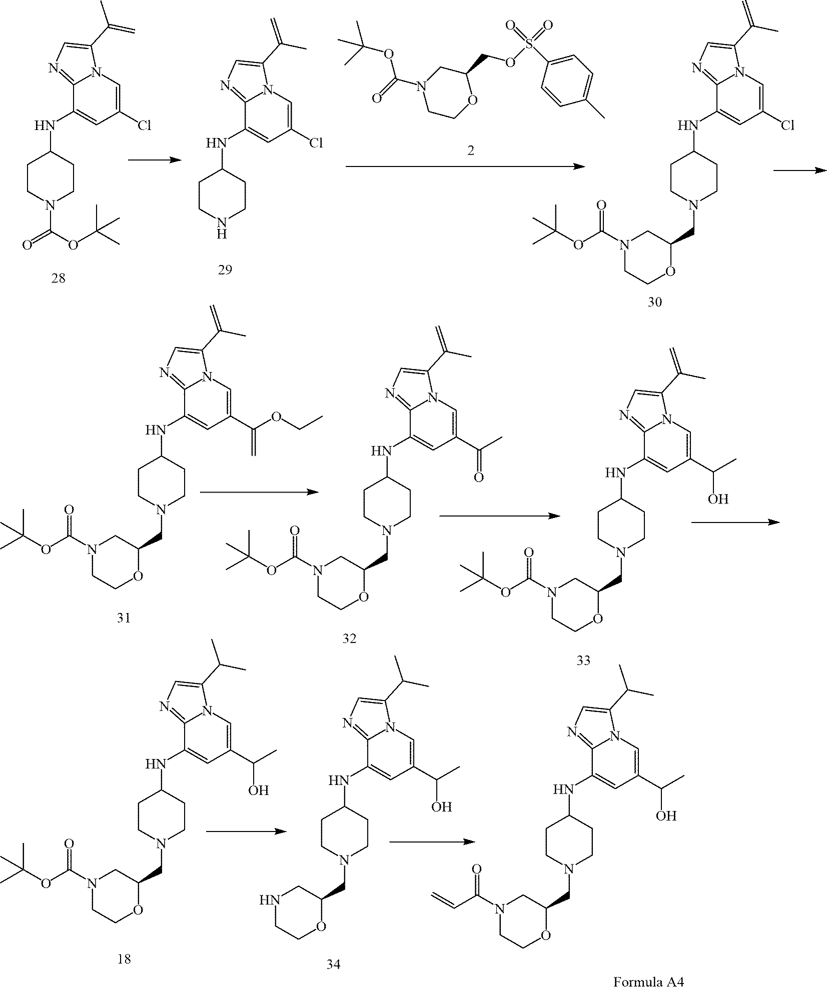

Scheme 7 depicts the synthesis of the compounds of Formula A4. Deprotection of 28 and subsequent substitution with N-protected morpholinosulfonate 2 may be carried out essentially as described in scheme 5. Addition of the enol ether followed by hydrolysis and reduction to the secondary alcohol 33 may be carried out essentially as described in Scheme 4. Reduction of isopropenyl imidazopyridine 33 may be carried out essentially as described in Scheme 3. Deprotection of N-protected morpholino 18 and formation of acrylamide Formula A4 may be carried out essentially as described in Scheme 3.

### Preparation 1

N-(1,1-difluoroethyl)-2-pyridyl]-1, 1-diphenyl-methanimine

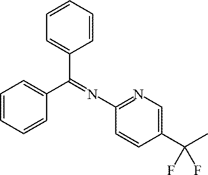 [SMILES: CC(F)(F)c1ccc(N=C(c2ccccc2)c2ccccc2)nc1]

Add diphenylmethanimine (9.5 g, 52 mmol), (rac)-2,2′-bis(diphenylphosphino)-1,1′-binaphthyl (4.2 g, 6.5 mmol), and C_{S2}CO₃ (18.5 g, 57 mmol) to a solution of 2-bromo-5-(1,1-difluoroethyl)pyridine (10 g, 44 mmol) in toluene (175 mL). Add palladium(II) acetate (0.98 g, 4.4 mmol), purge with N₂ and heat at 100° C. After 16 h, filter through a pad of diatomaceous earth and wash with EtOAc (400 mL). Remove solvent under reduced pressure to afford a brown oil. Purify the residue by column chromatography eluting with EtOAc: hexanes (0-30% gradient). Combine appropriate fractions and concentrate under reduced pressure to give N-[5-(1,1-difluoroethyl)-2-pyridyl]-1,1-diphenyl-methanimine. Following this preparation gave 8.2 g (49% yield) as a yellow oil. ES/MS (m/z): 323 (M+H).

### Preparation 2

5-(1,1-difluoroethyl)pyridin-2-amine

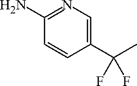

Add HCl (5 M in IPA, 13 mL, 67 mmol,) to a solution of N-[5-(1,1-difluoroethyl)-2-pyridyl]-1,1-diphenyl-methanimine (8.6 g, 27 mmol) in DCM (134 mL) and MeOH (134 mL). Stir at RT for 1 h. Evaporate the solvent and sonicate the residue with hexanes/MTBE (9:1) (50 mL). Decant the solid and wash with more solvent mixture (2 × 50 mL). Treat the solid with 2 N NH₃ in MeOH (40 mL). Evaporate the solvents under reduced pressure to give 5-(1,1-difluoroethyl)pyridin-2-amine. Following this preparation gave 4.77 g (96% yield) as an oily white solid. ES/MS (m/z): 159 (M+H). ¹H NMR (400.13 MHz, DMSO): 8.10 (dd, J= 0.9, 2.3 Hz, 1H), 7.54-7.49 (m, 1H), 6.47 (dd, J= 0.6, 8.7 Hz, 1H), 6.31 (bs, 2H), 1.93 (t, J= 18 Hz, 3H).

### Preparation 3

3-chloro-5-(1,1-difluoroethyl)pyridin-2-amine

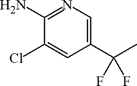

Add NCS (1.3 g, 9.8 mmol) portionwise over 20 minutes, to a solution of 5-(1,1-difluoroethyl)pyridin-2-amine (1.5 g, 6.5 mmol) in ACN (26 mL) and stir at RT for 3 days. Remove volatiles under reduced pressure and purify the residue on a SCX column (50 g): 2 volumes MeOH. Dissolve the crude material in DCM (5 × 3 mL) and load into the column. Wash first with DCM, then with MeOH and elute with 7 M NH₃ in MeOH (250 mL). Evaporate the basic fraction to afford 3-chloro-5-(1,1-difluoroethyl)pyridin-2-amine. Following this preparation gave 1.17 g (84% yield) as a dark-brown oil. ES/MS m/z (³⁵Cl/³⁷Cl) 193/195. ¹H NMR (400.13 MHz, DMSO): 8.12-8.11 (m, 1H), 7.76 (d, J= 2.1 Hz, 1H), 6.72 (s, 2H), 1.96 (t, J= 18 Hz, 3H).

### Preparation 4

8-chloro-6-(1,1-difluoroethyl)imidazo[1,2-a]pyridine

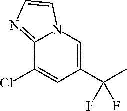

Treat a solution of 3-chloro-5-(1,1-difluoroethyl)pyridin-2-amine (4.5 g, 19 mmol) in EtOH (95 mL) with 2-chloroacetaldehyde (55 mass%, 8.9 mL, 76 mmol). Reflux the reaction for 2.5 h. Evaporate the EtOH and treat the residue with saturated aqueous NaHCO₃ and extracted with DCM (2 × 80 mL). Dry organic phase over anhydrous Na₂SO₄, filter and concentrate under reduced pressure to afford a dark-brown oil. Purify the residue by column chromatography eluting with EtOAc: hexanes (0-60% gradient). Combine appropriate fractions and concentrate under reduced pressure to give 8-chloro-6-(1,1-difluoroethyl)imidazo[1,2-a]pyridine. Following this preparation gave 1.48 g (36% yield) as a brown oil. ES/MS m/z (³⁵Cl/³⁷Cl) 217/219. ¹H NMR (400.13 MHz, DMSO): 8.95 (q, J= 1.5 Hz, 1H), 8.15 (d, J= 1.3 Hz, 1H), 7.72 (d, J= 1.3 Hz, 1H), 7.64 (d, J= 1.5 Hz, 1H), 2.06 (t, J= 19 Hz, 3H).

### Preparation 5

8-chloro-6-(1,1-difluoroethyl)-3-iodo-imidazo[1,2-a]pyridine

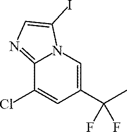

Add NIS (1.7 g, 7.4 mmol) to a solution of 8-chloro-6-(1,1-difluoroethyl)imidazo[1,2-a]pyridine (1.48 g, 6.7 mmol) in ACN (34 mL) and stir at RT for 16 h. Evaporate all volatiles, dissolve crude material in 2-MeTHF (350 mL), wash with 1 M Na₂S2O₃ (1 × 50 mL) and saturated aqueous NaHCO₃ (3 × 50 mL). Dry organic layer over anhydrous Na₂SO₄, filter and evaporate to give a brown oil. Purify the residue by column chromatography eluting with EtOAc: hexanes (0-35% gradient). Combine appropriate fractions and concentrate under reduced pressure to give 8-chloro-6-(1,1-difluoroethyl)-3-iodo-imidazo[1,2-a]pyridine. Following this preparation gave 1.9 g (81% yield) as a light-brown solid. ES/MS m/z (³⁵C1/³⁷Cl) 343/345. ¹H NMR (400.13 MHz, DMSO): 8.36 (q, J= 1.5 Hz, 1H), 7.89 (s, 1H), 7.78 (d, J= 1.5 Hz, 1H), 2.11 (t, J= 19 Hz, 3H).

### Preparation 6

8-chloro-6-(1,1-difluoroethyl)-3-isopropenyl-imidazo[1,2-a]pyridine

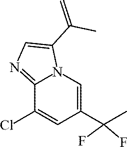 [SMILES: C=C(C)c1cnc2c(Cl)cc(C(C)(F)F)cn12]

Dissolve 8-chloro-6-(1,1-difluoroethyl)-3-iodo-imidazo[1,2-a]pyridine (2.2 g, 6.4 mmol) in EtOH (43 mL). Add 1.2 M K₂CO₃ in water (16 mL, 19.3 mmol) under N₂. Purge with N₂ for 5 min with an outlet needle. Add 2-isopropenyl-4,4,5,5-tetramethyl-1,3,2-dioxaborolane (1.25 g, 7.1 mmol) and BrettPhos Pd G3 (0.3 g, 0.32 mmol). Purge again with N₂ for 5 min. and stir at RT for 20 h. Remove volatiles and partition the residue between 2-MeTHF (22 mL) and water (11 mL). Further extract the aqueous layer with 2-MeTHF (22 mL), dry organics over anhydrous Na₂SO₄, filter and evaporate all volatiles to give a brown oil. Purify the residue by column chromatography eluting with EtOAc: hexanes (0-40% gradient). Combine appropriate fractions and concentrate under reduced pressure to afford 8-chloro-6-(1,1-difluoroethyl)-3-isopropenyl-imidazo[1,2-a]pyridine. Following this preparation gave 1.49 g (90% yield) as a light-brown oil. ES/MS m/z (³⁵Cl/³⁷Cl) 257/259. ¹H NMR (400.21 MHz, DMSO): 8.61 (q, J= 1.5 Hz, 1H), 7.86 (s, 1H), 7.70 (d, J= 1.5 Hz, 1H), 5.51 (s, 1H), 5.47 (dd, J= 0.7, 1.4 Hz, 1H), 2.09 (t, J= 19 Hz, 3H).

### Preparation 7

8-chloro-6-(1,1-difluoroethyl)-3-isopropyl-imidazo[1,2-a]pyridine

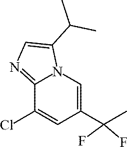 [SMILES: CC(C)c1cnc2c(Cl)cc(C(C)(F)F)cn12]

Dissolve 8-chloro-6-(1,1-difluoroethyl)-3-isopropenyl-imidazo[1,2-a]pyridine (1.49 g, 5.80 mmol) in MeOH (41 mL). Add platinum (type 128 M, 5.34% Pt (dry weight basis) with 58% moisture, 1.06 g, 0.12 mmol) under N₂. Stir under H₂ atmosphere (balloon) for 80 min. Filter through a pad of diatomaceous earth eluting with a 1:1 MeOH/EtOH mixture (100 mL). Remove all volatiles under reduced pressure to obtain 8-chloro-6-(1,1-difluoroethyl)-3-isopropyl-imidazo[1,2-a]pyridine. Following this preparation gave 1.47 g (91% yield) as a light-yellow oil. ES/MS m/z (³⁵C1/³⁷Cl) 259/261. ¹H NMR (400.13 MHz, DMSO): 8.52 (q, J= 1.5 Hz, 1H), 7.61 (d, J= 1.5 Hz, 1H), 7.56 (d, J= 0.7 Hz, 1H), 2.10 (t, J= 19 Hz, 3H), 1.33 (d, J= 6.8 Hz, 6H).

### Preparation 8

6,8-dichloro-3-iodo-imidazo[1,2-a]pyridine

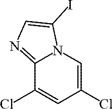 [SMILES: Clc1cc(Cl)c2ncc(I)n2c1]

Add NIS (70.3 g, 306 mmol) to a solution of 6,8-dichloroimidazo[1,2-a]pyridine (52.1 g, 278.5 mmol) in ACN (1.4 L ) and stir at RT for 30 h. Filter the suspension and wash the solid with ACN. Dry under a stream of air to afford 6,8-dichloro-3-iodo-imidazo[1,2-a]pyridine (54.4 g, 62% yield) as a pale brown solid. Evaporate the mother liquor under reduced pressure. Dissolve the crude in in 2-MeTHF (520 mL), wash with Na₂S₂O₃ (25 % w/v) (520 mL) and with NaHCO₃ (9% w/v) (520 mL). Separate the organic phase, dry over anhydrous MgSO₄ and concentrate under reduced pressure to afford 6,8-dichloro-3-iodo-imidazo[1,2-a]pyridine. Following this preparation gave 30.4 g (35% yield) as a white solid. ES/MS (m/z): (³⁵C1/³⁷Cl) 312/314. ¹H NMR (400.21 MHz, CDCl₃): 8.17 (d, J= 1.7 Hz, 1H), 7.79 (s, 1H), 7.38 (d, J= 1.7 Hz, 1H).

### Preparation 9

6,8-dichloro-3-isopropenyl-imidazo[1,2-a]pyridine

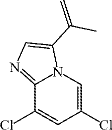

In a high pressure tube add 6,8-dichloro-3-iodo-imidazo[1,2-a]pyridine (54.9 g, 175.7 mmol), 1,4-dioxane (1.1 L) and 1.2 M K₂CO₃ in water (440 mL, 527 mmol). Purge the mixture with a N₂ stream (three times), add 2-isopropenyl-4,4,5,5-tetramethyl-1,3,2-dioxaborolane (34.2 g, 193 mmol), Brettphos Pd G3 (4.06 g, 4.39 mmol) and purge again (3×). Cap the tube and heat the mixture at 50° C. for 26 h. Remove volatiles under reduced pressure. Suspend the residue in 2-MeTHF (550 mL) and water (275 mL). Separate the organic phase, dry over anhydrous MgSO₄, and concentrate under reduced pressure. Purify the residue by flash chromatography eluting with EtOAc: hexanes (0-40% gradient). Combine appropriate fractions and concentrate under reduced to afford 6,8-dichloro-3-isopropenyl-imidazo[1,2-a]pyridine. Following this preparation gave 36.4 g (87% yield) as a yellow solid. ES/MS (m/z): (³⁵C1/³⁷Cl) 227/229.

### Preparation 10

6,8-dichloro-3-isopropyl-imidazo[1,2-a]pyridine

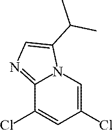

Stir a solution of 6,8-dichloro-3-isopropenyl-imidazo[1,2-a]pyridine (23.6 g, 98.7 mmol), platinum (18.1 g, 2.0 mmol), and MeOH (592 mL) at RT under H₂ for 7 h. Filter the mixture through a pad of diatomaceous earth, rinse with MeOH and concentrate under reduced pressure. Triturate the crude material with 288 mL of water overnight. Filter under reduced pressure using a sinter funnel (3 Å pore size). Dry under a stream of air and under high vacuum overnight to afford 6,8-dichloro-3-isopropyl-imidazo[1,2-a]pyridine. Following this preparation gave 14.9 g (63% yield) as a white solid. ES/MS (m/z): (³⁵Cl/³⁷Cl) 229/231. ¹H NMR (400.13 MH_{z}, CDCl₃): 7.94 (d, J= 1.7 H_{z}, 1H), 7.50 (d, J= 0.6 Hz, 1H), 7.27 (d, J= 1.8 Hz, 1H), 3.19-3.12 (m, 1H), 1.42 (d, J= 6.8 Hz, 6H).

### Preparation 11

tert-butyl (2R)-2-[[4-[(6-chloro-3-isopropyl-imidazo[1,2-a]pyridin-8-yl)amino]-1-piperidyl]methyl]morpholine-4-carboxylate

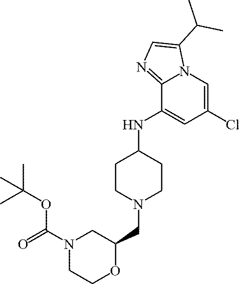 [SMILES: CC(C)c1cnc2c(NC3CCN(C[C@@H]4CN(C(=O)OC(C)(C)C)CCO4)CC3)cc(Cl)cn12]

In a high pressure tube add 6,8-dichloro-3-isopropyl-imidazo[1,2-a]pyridine (1.0 g, 4.17 mmol), tert-butyl (2R)-2-[(4-amino-1-piperidyl)methyl]morpholine-4-carboxylate (1.9 g, 6.25 mmol), sodium tert-butoxide (1.24 g, 12.5 mmol), and 1,4-dioxane (21 mL). Bubble N₂ to the solution and add BrettPhos Pd G3 (0.24 g, 0.25 mmol). Cap the tube and heat the reaction mixture at 100° C. under N₂ for 22 h. Cool the reaction mixture to room temperature, dilute with MTBE and wash with water. Separate the organic phase and extract the aqueous phase with MTBE (twice). Combine organic layers, dry over anhydrous Na₂SO₄ and concentrate under reduced pressure. Purify the crude material by flash chromatography eluting with MeOH: DCM (0-3% gradient). Concentrate under reduced pressure appropriate fractions and dry under high vacuo to afford tert-butyl (2R)-2-[[4-[(6-chloro-3-isopropyl-imidazo[1,2-a]pyridin-8-yl)amino]-1-piperidyl]methyl]morpholine-4-carboxylate. Following this preparation gave 1.37 g (66% yield) as a greenish foam. ES/MS (m/z): 492 (M+H). ¹H NMR (400.21 MHz, DMSO): 7.74 (d, J= 1.7 Hz, 1H), 7.22 (s, 1H), 6.14 (d, J= 1.5 Hz, 1H), 5.94 (d, J= 8.3 Hz, 1H), 3.86-3.68 (m, 3H), 3.49-3.41 (m, 3H), 3.26-3.20 (m, 1H), 2.87-2.79 (m, 3H), 2.40-2.31 (m, 2H), 2.23-2.10 (m, 2H), 1.90-1.87 (m, 2H), 1.62-1.50 (m, 2H), 1.41 (s, 9H), 1.28 (d, J= 6.8 Hz, 6H).

### Preparation 12

tert-butyl (2R)-2-[[4-[(6-acetyl-3-isopropyl-imidazo[1,2-a]pyridin-8-yl)amino]-1-piperidyl]methyl]morpholine-4-carboxylate

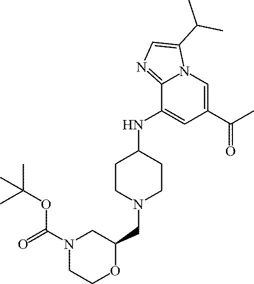 [SMILES: CC(=O)c1cc(NC2CCN(C[C@@H]3CN(C(=O)OC(C)(C)C)CCO3)CC2)c2ncc(C(C)C)n2c1]

Add tributyl(1-ethoxyvinyl)stannane (0.86 mL, 2.5 mmol,), CsF (0.59 g, 3.9 mmol), and XPhos- Pd G2 (0.15 g, 0.19 mmol) to a solution of tert-butyl (2R)-2-[[4-[(6-chloro-3-isopropyl-imidazo[1,2-a]pyridin-8-yl)amino]-1-piperidyl]methyl]morpholine-4-carboxylate (1.0 g, 1.9 mmol) in toluene (10 mL). Purge the solution with N₂ and stir at 95° C. for 4 h. Cool to room temperature, filter the mixture through a pad of diatomaceous earth, rinse with EtOAc, and concentrate under reduced pressure. Re-dissolve the residue in 2-propanol (19 mL). Add HCl (0.2 M in water) (19 mL) and stir at RT for 5.5 h. Neutralize with saturated aqueous NaHCO₃ solution. Add EtOAc and stir for 10 min. Separate the organic layer and extract the aqueous phase with additional EtOAc. Combine organic layers, dry over anhydrous Na₂SO₄, and concentrate under reduced pressure. Purify the crude material by silica gel eluting first with DCM: hexanes (50% isocratic) and then with MeOH: DCM (0-3% gradient). Concentrate under reduced pressure, dry under high vacuo to afford tert-butyl (2R)-2-[[4-[(6-acetyl-3-isopropyl-imidazo [1,2-a]pyridin-8-yl)amino]-1-piperidyl]methyl]morpholine-4-carboxylate. Following this preparation gave 0.76 g (68 % yield) as a yellow foam solid. ES/MS (m/z): 500 (M+H). ¹H NMR (400.13 MHz, CDCl₃): 8.04 (d, J= 1.4 Hz, 1H), 7.30 (d, J= 0.7 Hz, 1H), 6.60 (d, J= 1.1 Hz, 1H), 5.22-5.16 (m, 1H), 4.01-4.00 (m, 3H), 3.63-3.58 (m, 3H), 3.26-3.20 (m, 1H), 2.97-2.89 (m, 3H), 2.62 (s, 5H), 2.41-2.34 (m, 3H), 2.19-2.11 (m, 2H), 1.82-1.80 (m, 2H), 1.49 (s, 9H), 1.44 (d, J= 6.9 Hz, 6H).

### Preparation 13

Rac-tert-butyl (2R)-2-[[4-[[6-(1-hydroxyethyl)-3-isopropyl-imidazo[1,2-a]pyridin-8-yl]amino]-1-piperidyl] methyl]morpholine-4-carboxylate

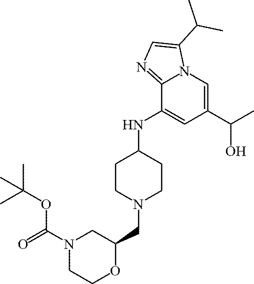 [SMILES: CC(C)c1cnc2c(NC3CCN(C[C@@H]4CN(C(=O)OC(C)(C)C)CCO4)CC3)cc(C(C)O)cn12]

Cool to 0° C. a solution of tert-butyl (2R)-2-[[4-[(6-acetyl-3-isopropyl-imidazo[1,2-a]pyridin-8-yl)amino]-1-piperidyl]methyl]morpholine-4-carboxylate (460 mg, 0.79 mmol) in MeOH (4 mL) under N₂. Add NaBH₄ (0.04 g, 0.9 mmol) in portions and stir the mixture at RT for 15 min. Dilute with MeOH and add water slowly. Remove the organic solvent under reduced pressure. Dilute the residue with EtOAc and wash with water. Combine organic layers, dry over anhydrous MgSO₄, filter, and concentrate under reduce pressure to afford the title compound. Following this preparation gave 0.4 g (88% yield) as pale brown solid. ES/MS (m/z): 502 (M+H). ¹H NMR (400.21 MHz, CDCl₃): 7.38 (s, 1H), 7.22 (d, J= 0.7 Hz, 1H), 6.09 (d, J= 0.7 Hz, 1H), 5.12 (d, J= 7.9 Hz, 1H), 4.89 (q, J= 6.4 Hz, 1H), 3.97-3.86 (m, 3H), 3.60-3.50 (m, 3H), 3.19-3.12 (m, 1H), 2.94 (d, J= 9.5 Hz, 3H), 2.72-2.56 (m, 2H), 2.37-2.25 (m, 3H), 2.13-2.10 (m, 2H), 1.74-1.64 (m, 4H), 1.57 (d, J= 6.4 Hz, 3H), 1.49 (s, 9H), 1.39 (d, J= 6.8 Hz, 3H).

### Preparation 14

Rac-tert-butyl (2R)-2-[[4-[[6-[1-fluoroethyl]-3-isopropyl-imidazo[1,2-a]pyridin-8-yl]amino]-1-piperidyl]methyl]morpholine-4-carboxylate

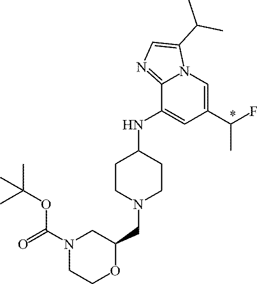

Charge a teflon tube with tert-butyl (2R)-2-[[4-[[6-(1-hydroxyethyl)-3-isopropyl-imidazo[1,2-a]pyridin-8-yl]amino]-1-piperidyl]methyl]morpholine-4-carboxylate (406 mg, 0.7 mmol) and DCM (2.3 mL) under N₂ and cool to -78° C. Add sequentially TEA (0.1 mL, 0.7 mmol), trimethylamine trihydrofluoride (0.2 mL, 1.4 mmol), and Xtalfluoro-E (279 mg, 1.1 mmol). Stir the mixture at -78° C. for 30 min. and then allow to warm to RT and stir for 20 h. Ice-cool the mixture and quench by the slow addition of saturated aqueous NaHCO₃, water and DCM. Extract further the aqueous layer with DCM. Combine organic layers, dry over anhydrous MgSO₄, filter, and concentrate under reduce pressure. Purify the crude material by silica gel eluting with MeOH: DCM (0-5% gradient). Concentrate under reduced pressure and dry under high vacuo to afford the racemic tert-butyl (2R)-2-[[4-[[6-[1-fluoroethyl]-3-isopropyl-imidazo[1,2-a]pyridin-8-yl]amino]-1-piperidyl]methyl]morpholine-4-carboxylate (chiral at asterisk). Following this preparation gave 0.23 g (60% yield) as a brown solid. ES/MS (m/z): 504 (M+H).

### Preparation 15 & 16

Isomer 1 - tert-butyl (2R)-2-[[4-[[6-(1-fluoroethyl)-3-isopropyl-imidazo[1,2-a]pyridin-8-yl]amino]-1-piperidyl]methyl]morpholine-4-carboxylate

Isomer 2 - tert-butyl (2R)-2-[[4-[[6-(1-fluoroethyl)-3-isopropyl-imidazo[1,2-a]pyridin-8-yl]amino]-1-piperidyl]methyl]morpholine-4-carboxylate

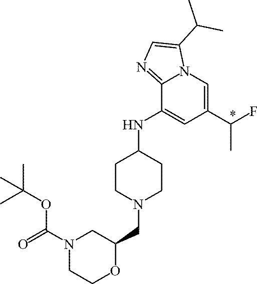

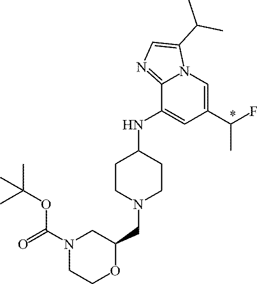

Purify racemic tert-butyl (2R)-2-[[4-[[6-[1-fluoroethyl]-3-isopropyl-imidazo[1,2-a]pyridin-8-yl]amino]-1-piperidyl]methyl]morpholine-4-carboxylate (0.23 g, 0.4 mmol). [Instrument: SFC10 (Sepiatec); Column: Chiralpak IG (25 × 2 cm, 5 um); Mobile phase: CO₂ (A)/IPA (0.2%DMEA) (B); Elution program: Isocratic 40% B; Outlet pressure: 100 bar; Column temperature: 40° C. ; Flow rate: 65 mL/min; Detection: UV at 220 nm to afford both separated enantiomers:
Isomer 1: Following this method, 72 mg of tert-butyl (2R)-2-[[4-[[6-(1-fluoroethyl)-3-isopropyl-imidazo[1,2-a]pyridin-8-yl]amino]-1-piperidyl]methyl]morpholine-4-carboxylate was obtained (19% yield) was obtained as a brown solid. (Achiral purity by RP-LC/MS, Rt = 1.3 min, 92%). ES/MS (m/z): 504 (M+H). (Chiral analysis, Rt = 1.1 min, e.e. >98%).
Isomer 2: Following this method, 105 mg of tert-butyl (2R)-2-[[4-[[6-(1-fluoroethyl)-3-isopropyl-imidazo[1,2-a]pyridin-8-yl]amino]-1-piperidyl]methyl]morpholine-4-carboxylate (27% yield) was obtained as a brown solid. (Achiral purity by RP-LC/MS, Rt = 1.3 min, 90%). ES/MS (m/z): 504 (M+H). (Chiral analysis, Rt = 1.4 min, e.e. >98%).
While the Isomer 1 and Isomer 2 enantiomers of Preparation 15 & 16 were separated, the specific chirality of each enantiomer at the asterisk position was not determined.

### Preparation 17

Isomer 1 - 6-(1-fluoroethyl)-3-isopropyl-N-[1-[[(2S)-morpholin-2-yl]methyl]-4-piperidyl]imidazo[1,2-a]pyridin-8-amine

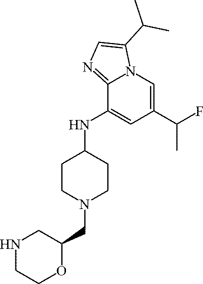 [SMILES: CC(C)c1cnc2c(NC3CCN(C[C@@H]4CNCCO4)CC3)cc(C(C)F)cn12]

Add 4 M HCl in dioxane (0.32 mL, 1.3 mmol) to a solution of Isomer 1 - tert-butyl (2R)-2-[[4-[[6-(1-fluoroethyl)-3-isopropyl-imidazo[1,2-a]pyridin-8-yl]amino]-1-piperidyl]methyl]morpholine-4-carboxylate (72 mg, 0.13 mmol) in DCM (1.3 mL) and stir at RT for 1 h. Remove volatiles under reduced pressure and purify the residue in a SCX column (10 g): 2 volumes MeOH. Dissolve the crude material in MeOH and load into the column, wash with MeOH and elute with 2 M NH₃ in MeOH. Evaporate the basic fraction to afford Isomer 1 - 6-(1-fluoroethyl)-3-isopropyl-N-[1-[[(2S)-morpholin-2-yl]methyl]-4-piperidyl]imidazo[1,2-a]pyridin-8-amine. Following this preparation gave 55 mg (98% yield) as a white solid. ES/MS (m/z): 404 (M+H).

### Example 1

Isomer 1 - 1-[(2R)-2-[[4-[[6-(1-fluoroethyl)-3-isopropyl-imidazo[1,2-a]pyridin-8-yl]amino]-1 -piperidyl]methyl]morpholin-4-yl]prop-2-en-1 -one

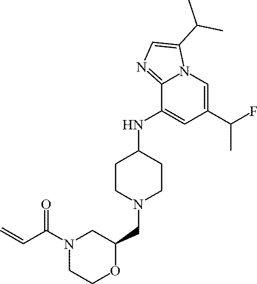 [SMILES: C=CC(=O)N1CCO[C@H](CN2CCC(Nc3cc(C(C)F)cn4c(C(C)C)cnc34)CC2)C1]

Add dropwise acryloyl chloride (0.009 ml, 0.118 mmol) to a cold solution (icebath) of Isomer 1 - 6-(1-fluoroethyl)-3-isopropyl-N-[1-[[(2S)-morpholin-2-yl]methyl]-4-piperidyl]imidazo[1,2-a]pyridin-8-amine (56 mg, 0.131 mmol) and TEA (0.07 ml, 0.527 mmol) in DCM (1.3 mL) and stir the mixture at this temperature for 30 min. Quench the reaction mixture with saturated aqueous NaHCO₃, stir at RT for 5 min, add water, and extract with DCM. Separate and combine organic phases, dry over anhydrous MgSO₄, filter, and concentrate under reduced pressure. Purify the crude material by silica gel eluting with MeOH: DCM (0-6% gradient). Concentrate under reduced pressure and dry under high vacuo to afford Isomer 1 - 1-[(2R)-2-[[4-[[6-(1-fluoroethyl)-3-isopropyl-imidazo[1,2-a]pyridin-8-yl]amino]-1-piperidyl]methyl]morpholin-4-yl]prop-2-en-1-one. Following this preparation gave 20 mg (31% yield) as a white solid. ES/MS (m/z): 458 (M+H). ¹H NMR (400.21 MHz, CDCl₃): 7.38 (s, 1H), 7.25 (s, 1H), 6.62-6.60 (m, 1H), 6.35 (dd, J= 1.7, 16.8 Hz, 1H), 6.09-6.07 (m, 1H), 5.77-5.74 (m, 1H), 5.70-5.55 (m, 1H), 4.60-4.56 (m, 1H), 4.01-3.95 (m, 2H), 3.69-3.65 (m, 3H), 3.34-3.32 (m, 5H), 2.66-2.61 (m, 7H), 1.75-1.68 (m, 5H), 1.40 (dd, J= 0.5, 6.8 Hz, 6H).

### Preparation 18

Isomer 2 - 6-(1-fluoroethyl)-3-isopropyl-N-[1-[[(2S)-morpholin-2-yl]methyl]-4-piperidyl]imidazo[1,2-a]pyridin-8-amine

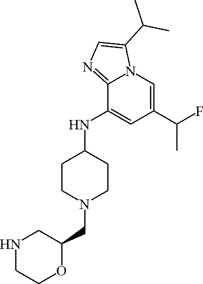 [SMILES: CC(C)c1cnc2c(NC3CCN(C[C@@H]4CNCCO4)CC3)cc(C(C)F)cn12]

Add 4 M HCl in dioxane (0.4 mL, 1.8 mmol) to a solution of Isomer 2 - tert-butyl (2R)-2-[[4-[[6-(1-fluoroethyl)-3-isopropyl-imidazo[1,2-a]pyridin-8-yl]amino]-1-piperidyl]methyl]morpholine-4-carboxylate (90 mg, 0.18 mmol) in DCM (1.8 mL) and stir at RT for 1 h. Remove volatiles under reduced pressure and purify the residue in a SCX column (10 g): 2 volumes MeOH. Dissolve the crude material in MeOH and load into the column, wash with MeOH and elute with 2 M NH₃ in MeOH. Evaporate the basic fraction to afford Isomer 2 - 6-(1-fluoroethyl)-3-isopropyl-N-[1-[[(2S)-morpholin-2-yl]methyl]-4-piperidyl]imidazo[1,2-a]pyridin-8-amine. Following this preparation gave 75 mg (98% yield) as a pale brown solid. ES/MS (m/z): 404 (M+H).

### Example 2

Isomer 2 - 1-[(2R)-2-[[4-[[6-(1-fluoroethyl)-3-isopropyl-imidazo[1,2-a]pyridin-8-yl]amino]-1 -piperidyl]methyl]morpholin-4-yl]prop-2-en-1 -one

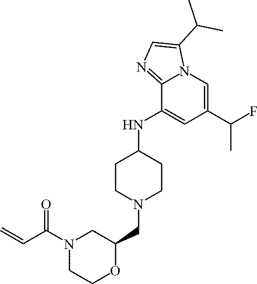 [SMILES: C=CC(=O)N1CCO[C@H](CN2CCC(Nc3cc(C(C)F)cn4c(C(C)C)cnc34)CC2)C1]

Add dropwise acryloyl chloride (0.012 ml, 0.159 mmol) to a cold solution (icebath) of Isomer 2 - 6-(1-fluoroethyl)-3-isopropyl-N-[1-[[(2S)-morpholin-2-yl]methyl]-4-piperidyl]imidazo[1,2-a]pyridin-8-amine (75 mg, 0.176 mmol) and TEA (0.098 mL, 0.706 mmol) in DCM (1.7 mL) and stir the mixture at this temperature for 30 min. Quench the reaction mixture with saturated aqueous NaHCO₃, stir at RT for 5 min add water and extract with DCM. Separate and combine organic phases and dry over anhydrous MgSO₄. Filter and concentrate under reduced pressure. Purify the crude material by silica gel eluting with MeOH: DCM (0-6% gradient). Concentrate under reduced pressure and dry under high vacuo to afford Isomer 2 - 1-[(2R)-2-[[4-[[6-(1-fluoroethyl)-3-isopropyl-imidazo[1,2-a]pyridin-8-yl]amino]-1-piperidyl]methyl]morpholin-4-yl]prop-2-en-1-one. Following this preparation gave 28 mg (33% yield) as a pale brown solid. ES/MS (m/z): 458 (M+H). ¹H NMR (400.13 MHz, CDCl₃): 7.37 (s, 1H), 7.25 (s, 1H), 6.66-6.61 (m, 1H), 6.34 (dd, J= 1.7, 16.8 Hz, 1H), 6.06 (s, 1H), 5.77-5.57 (m, 2H), 4.61-4.57 (m, 1H), 4.00-3.95 (m, 2H), 3.69-3.65 (m, 3H), 3.33-3.32 (m, 5H), 2.67-2.62 (m, 7H), 1.75-1.68 (m, 5H), 1.40 (d, J= 6.9 Hz, 6H).

### Preparation 19

tert-butyl 4-[(6-chloro-3-isopropenyl-imidazo[1,2-a]pyridin-8-yl)amino]piperidine-1-carboxylate

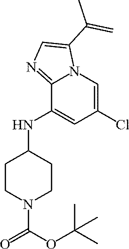 [SMILES: C=C(C)c1cnc2c(NC3CCN(C(=O)OC(C)(C)C)CC3)cc(Cl)cn12]

Charge a high pressure vessel with 6,8-dichloro-3-isopropenyl-imidazo[1,2-a]pyridine (3.42 g, 12.3 mmol), 1,4-dioxane (84 mL), tert-butyl 4-aminopiperidine-1-carboxylate (3.05 g, 15.2 mmol), and sodium tert-butoxide (3.65 g, 38.0 mmol). Bubble N₂ into the solution and add Brettphos Pd G3 (940 mg, 1.02 mmol). Bubble N₂ on to the resulting mixture again, cap the tube and heat the reaction mixture at 95° C. under N₂ for 2 h. Cool the reaction mixture to room temperature, dilute with EtOAc, and wash with saturated aqueous NaHCO₃. Separate the organic phase and wash saturated aqueous NaCl, dry over anhydrous MgSO₄, filter, and concentrate under reduced pressure. Purify the residue by flash chromatography, eluting with a mixture MTBE: hexanes (10-60% gradient), followed by acetone: hexanes (10-40% gradient). Concentrate under reduced pressure and dry to afford tert-butyl 4-[(6-chloro-3-isopropenyl-imidazo[1,2-a]pyridin-8-yl)amino]piperidine-1-carboxylate. Following this preparation gave 3.16 g (62.8% yield) as a yellow oil. ES/MS (m/z): 391 (M+H). ¹H NMR (400.13 MHz, DMSO): 7.88 (d, J= 1.7 Hz, 1H), 7.56 (s, 1H), 6.33 (d, J= 1.7 Hz, 1H), 6.20 (d, J= 8.8 Hz, 1H), 5.35 (d, J= 30.3 Hz, 2H), 3.95 (d, J= 12.8 Hz, 2H), 3.72-3.62 (m, 1H), 2.99-2.81 (m, 2H), 2.17 (s, 3H), 1.90 (dd, J= 2.0, 12.7 Hz, 2H), 1.58-1.37 (m, 2H), 1.42 (s, 9H).

### Preparation 20

6-chloro-3-isopropenyl-N-(4-piperidyl)imidazo[1,2-a]pyridin-8-amine

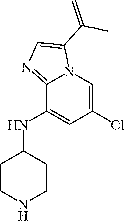 [SMILES: C=C(C)c1cnc2c(NC3CCNCC3)cc(Cl)cn12]

Add TFA (12 mL, 158.7 mmol) to a solution of tert-butyl 4-[(6-chloro-3-isopropenyl-imidazo[1,2-a]pyridin-8-yl)amino]piperidine-1-carboxylate (2.96 g, 7.57 mmol) in DCM (50 mL) and stir the mixture at RT for 1 h. Remove volatiles under reduced pressure and dissolve the residue in MeOH. Load the solution on to a SCX cartridge (50 g) pre-treated with MeOH. Elute with MeOH and MeOH (7N NH₃). Collect and concentrate the basic fractions in vacuo to yield 6-chloro-3-isopropenyl-N-(4-piperidyl)imidazo[1,2-a]pyridin-8-amine. Following this preparation gave 2.2 g (98.9% yield) as a green oil. ES/MS (m/z): 291(M+H). ¹H NMR (400.13 MHz, DMSO): 7.87 (d, J= 1.8 Hz, 1H), 7.55 (s, 1H), 6.26 (d, J= 1.5 Hz, 1H), 5.98 (d, J= 8.4 Hz, 1H), 5.35 (d, J= 29.7 Hz, 2H), 3.59-3.53 (m, 2H), 2.97-2.92 (m, 2H), 2.60 (td, J= 12.1, 2.1 Hz, 2H), 2.17 (d, J= 0.6 Hz, 3H), 1.90-1.87 (m, 2H), 1.58-1.37 (m, 2H).

### Preparation 21

tert-butyl (2R)-2-[[4-[(6-chloro-3-isopropenyl-imidazo[1,2-a]pyridin-8-yl)amino]-1-piperidyl]methyl]morpholine-4-carboxylate

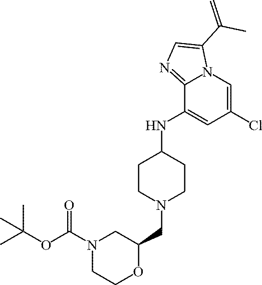 [SMILES: C=C(C)c1cnc2c(NC3CCN(C[C@@H]4CN(C(=O)OC(C)(C)C)CCO4)CC3)cc(Cl)cn12]

Add DIPEA (4 mL, 22.9 mmol) to a stirred solution of 6-chloro-3-isopropenyl-N-(4-piperidyl)imidazo[1,2-a]pyridin-8-amine (2.2 g, 7.5 mmol) and tert-butyl (2S)-2-(p-tolylsulfonyloxymethyl)morpholine-4-carboxylate (3.4 g, 9.2 mmol,) in anhydrous ACN (25 mL). Stir the mixture at 100° C. overnight. Cool down the reaction mixture to RT and evaporate the volatiles under reduced pressure. Purify the residue by flash chromatography with MeOH: DCM (0-10% gradient). Concentrate in vacuo to yield tert-butyl (2R)-2-[[4-[(6-chloro-3-isopropenyl-imidazo[1,2-a]pyridin-8-yl)amino]-1-piperidyl]methyl]morpholine-4-carboxylate. Following this preparation gave 2.18 g (59% yield) as a light green semisolid. ES/MS (m/z): 490 (M+H). ¹H NMR (400.13 MHz, CDCl₃): 7.80 (d, J= 1.7 Hz, 1H), 7.44 (s, 1H), 7.28 (s, 1H), 6.09 (d, J= 1.7 Hz, 1H), 5.34-5.25 (m, 3H), 4.02-3.97 (m, 3H), 3.60-3.52 (m, 3H), 2.95 (d, J= 8.3 Hz, 3H), 2.69-2.63 (m, 2H), 2.38-2.25 (m, 3H), 2.13-2.10 (m, 2H), 1.75-1.65 (m, 3H), 1.49 (s, 9H

### Preparation 22

tert-butyl (2R)-2-[[4-[[6-(1-ethoxyvinyl)-3-isopropenyl-imidazo[1,2-a]pyridin-8-yl]amino]-1-piperidyl]methyl]morpholine-4-carboxylate

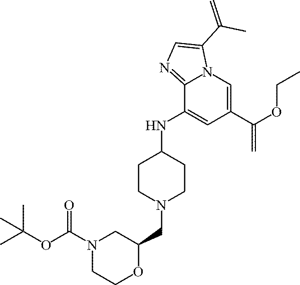 [SMILES: C=C(OCC)c1cc(NC2CCN(C[C@@H]3CN(C(=O)OC(C)(C)C)CCO3)CC2)c2ncc(C(=C)C)n2c1]

Add, to a microwave tube, a solution of tert-butyl (2R)-2-[[4-[(6-chloro-3-isopropenyl-imidazo[1,2-a]pyridin-8-yl)amino]-1-piperidyl]methyl]morpholine-4-carboxylate (0.42 g, 0.85 mmol) in toluene (17 mL), tributyl(1-ethoxyvinyl)tin (0.39 mL, 1.12 mmol), chloro(2-dicyclohexylphosphino-2′,4′,6′-tri-i-propyl-1,1′-biphenyl)(2′-amino-1,1′-biphenyl-2-yl) palladium(II) (75 mg, 0.093 mmol), and CsF (0.26 g, 1.71 mmol). Bubble N₂ to the reaction mixture for 5 min, cap the tube and heat at 100° C. for 3 h. Cool down to RT, add EtOAc to the crude mixture and filter through a pad of diatomaceous earth rinsing with EtOAc. Remove the volatiles under reduced pressure to afford tert-butyl (2R)-2-[[4-[[6-(1-ethoxyvinyl)-3-isopropenyl-imidazo[1,2-a]pyridin-8-yl]amino]-1-piperidyl]methyl]morpholine-4-carboxylate. Following this preparation gave 0.757 g (crude material) as a brown oil. ES/MS (m/z): 526 (M+H).

### Preparation 23

tert-butyl (2R)-2-[[4-[(6-acetyl-3-isopropenyl-imidazo[1,2-a]pyridin-8-yl)amino]-1-piperidyl]methyl]morpholine-4-carboxylate

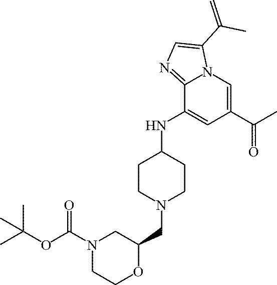 [SMILES: C=C(C)c1cnc2c(NC3CCN(C[C@@H]4CN(C(=O)OC(C)(C)C)CCO4)CC3)cc(C(C)=O)cn12]

Add HCl in water (4 mL, 0.2 M) to a solution of tert-butyl (2R)-2-[[4-[[6-(1-ethoxyvinyl)-3-isopropenyl-imidazo[1,2-a]pyridin-8-yl]amino]-1-piperidyl]methyl]morpholine-4-carboxylate (0.72 g, 0.82 mmol) in 2-propanol (1.5 mL). Stir the reaction mixture at room temperature for 1 h. Add EtOAc followed by a saturated aqueous solution of NaHCO₃ and stir the mixture for 1 h at room temperature. Separate the organic layer, wash with water, dry over anhydrous MgSO₄, filter, and concentrate under reduce pressure. Purify the residue by flash chromatograph eluting with EtOH: hexanes (20-50% gradient) to give tert-butyl (2R)-2-[[4-[(6-acetyl-3-isopropenyl-imidazo[1,2-a]pyridin-8-yl)amino]-1-piperidyl]methyl]morpholine-4-carboxylate. Following this preparation gave 0.25 g (58% yield) as a brown oil. ES/MS (m/z): 498 (M+H). ¹H NMR (400.13 MHz, DMSO): 8.50 (d, J= 1.3 Hz, 1H), 7.65 (s, 1H), 6.54 (d, J= 1.1 Hz, 1H), 5.87 (d, J= 8.6 Hz, 1H), 5.51 (s, 1H), 5.40 (s, 1H), 3.89-3.77 (m, 3H), 3.50-3.43 (m, 4H), 2.90-2.75 (m, 3H), 2.60 (s, 3H), 2.42-2.33 (m, 2H), 2.28-2.10 (m, 4H), 1.97-1.78 (m, 2H), 1.65-1.54 (m, 3H), 1.41 (s, 9H).

### Preparation 24

Rac-tert-butyl (2R)-2-[[4-[[6-(1-hydroxyethyl)-3-isopropenyl-imidazo[1,2-a]pyridin-8-yl]amino]-1-piperidyl]methyl]morpholine-4-carboxylate

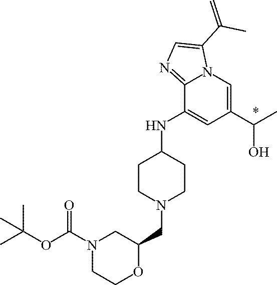

Add NaBH₄ (0.04 g, 1.03 mmol) to a solution of tert-butyl (2R)-2-[[4-[(6-acetyl-3-isopropenyl-imidazo[1,2-a]pyridin-8-yl)amino]-1-piperidyl]methyl]morpholine-4-carboxylate (0.39 g, 0.756 mmol) in EtOH (7.5 mL). Stir the reaction mixture at room temperature for 1 h. Add water followed by EtOAc to neutralize the excess NaBH4. Isolate the organic layer, dry over anhydrous MgSO₄, filter, and concentrate under reduce pressure to afford racemic tert-butyl (2R)-2-[[4-[[6-(1-hydroxyethyl)-3-isopropenyl-imidazo[1,2-a]pyridin-8-yl]amino]-1-piperidyl]methyl]morpholine-4-carboxylate (chiral at asterisk). Following this preparation gave 0.376 g (crude material, 94% yield) as a brown oil. ES/MS (m/z): 500 (M+H). ¹H NMR (400.13 MHz, DMSO): 7.83 (s, 1H), 7.50 (s, 1H), 6.21 (s, 1H), 5.54-5.18 (m, 4H), 4.75-4.69 (m, 1H), 4.09 (q, J= 5.3 Hz, 1H), 3.90-3.77 (m, 3H), 3.52-3.27 (m, 5H), 2.92-2.88 (m, 2H), 2.42-2.33 (m, 2H), 2.18-2.15 (m, 4H), 2.02-1.95 (m, 2H), 1.41-1.36 (m, 14H).

### Preparation 25

Rac-tert-butyl (2R)-2-[[4-[[6-(1-hydroxyethyl)-3-isopropyl-imidazo[1,2-a]pyridin-8-yl]amino]-1-piperidyl]methyl]morpholine-4-carboxylate

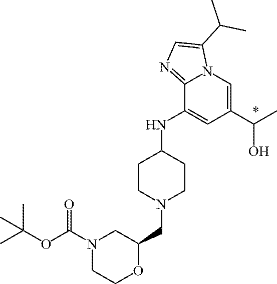

Add palladium (10 mass%) in Lindlar catalyst (0.25 g, 0.23 mmol) to a solution of racemic tert-butyl (2R)-2-[[4-[[6-(1-hydroxyethyl)-3-isopropenyl-imidazo[1,2-a]pyridin-8-yl]amino]-1-piperidyl]methyl]morpholine-4-carboxylate (0.29 g, 0.59 mmol) in MeOH (30 mL). Bubble N₂ in to the resulting mixture followed by three cycles of vacuum and H₂. Stir the reaction mixture under H₂ (1 atm) at room temperature for 5 h. Filter the reaction mixture through a pad of diatomaceous earth and wash thoroughly with MeOH. Remove volatiles under reduced pressure and dry to afford racemic tert-butyl (2R)-2-[[4-[[6-(1-hydroxyethyl)-3-isopropyl-imidazo[1,2-a]pyridin-8-yl]amino]-1-piperidyl]methyl]morpholine-4-carboxylate (chiral at asterisk). Following this preparation gave 0.18 g (55.6% yield) as a brown solid. ES/MS (m/z): 502 (M+H). ¹H NMR (400.21 MHz, DMSO): 7.47 (s, 1H), 7.15 (d, J= 0.7 Hz, 1H), 6.13 (s, 1H), 5.43-5.41 (m, 1H), 5.27-5.22 (m, 1H), 4.72-4.68 (m, 1H), 3.90-3.85 (m, 4H), 3.50-3.27 (m, 8H), 3.23-3.16 (m, 1H), 2.96-2.93 (m, 4H), 2.40-2.33 (m, 2H), 2.29-2.28 (m, 2H), 2.03-2.00 (m, 2H), 1.41-1.29 (m, 14H).

### Preparation 26

Rac[3-isopropyl-8-[[1-[[(2S)-morpholin-2-yl]methyl]-4-piperidyl]amino]imidazo[1,2-a]pyridin-6-yl]ethanol

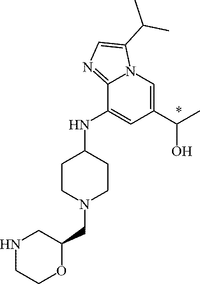

Add TFA (0.5 mL) to a solution of racemic tert-butyl (2R)-2-[[4-[[6-(1-hydroxyethyl)-3-isopropyl-imidazo[1,2-a]pyridin-8-yl]amino]-1-piperidyl]methyl]morpholine-4-carboxylate (160 mg, 0.319 mmol) in DCM (3 mL). Stir the reaction mixture at room temperature for 2 h. Evaporate the solvent under reduced pressure and purify the residue by SCX (10 g cartridge), elute with MeOH (3 CV), then 2N NH₃ in MeOH (3 CV). Combine the basic fractions and remove the solvent in vacuo to give racemic 1-[3-isopropyl-8-[[1-[[(2S)-morpholin-2-yl]methyl]-4-piperidyl]amino]imidazo[1,2-a]pyridin-6-yl]ethanol (chiral at asterisk). Following this preparation gave 120 mg (88% yield) as a brown solid. ES/MS (m/z): 402 (M+H). ¹H NMR (400.13 MHz, DMSO): 7.47 (s, 1H), 7.15 (s, 1H), 6.12 (s, 1H), 5.39 (d, J= 8.4 Hz, 1H), 5.18-5.11 (m, 1H), 4.74-4.65 (m, 1H), 3.72-3.63 (m, 1H), 3.50-3.45 (m, 4H), 3.23-3.14 (m, 2H), 2.90-2.73 (m, 2H), 2.70-2.63 (m, 2H), 2.43-2.41 (m, 4H), 2.04-2.01 (m, 2H), 1.62-1.55 (m, 2H), 1.38-1.29 (m, 10H).

### Example 3

Rac[(2R)-2-[[4-[[6-(1-hydroxyethyl)-3-isopropyl-imidazo[1,2-a]pyridin-8-yl]amino]piperidyl]methyl]morpholin-4-yl]prop-2-enone

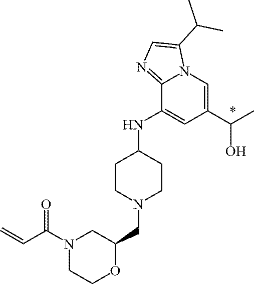

Add DIPEA (156 µL, 0.894 mmol) to a solution of racemic 1-[3-isopropyl-8-[[1-[[(2S)-morpholin-2-yl]methyl]-4-piperidyl]amino]imidazo[1,2-a]pyridin-6-yl]ethanol (120 mg, 0.299 mmol) in acetonitrile (5 mL). Cool the reaction mixture at 0° C. and add dropwise a solution of prop-2-enoyl chloride (25 µL, 0.307 mmol) in DCM (1.5 mL). Stir the resulting mixture at 0° C. for 60 min. Evaporate the solvent under reduced pressure. Treat the reaction mixture with a saturated solution of NaHCO₃ and extract with EtOAc. Wash the organic layer with water, dry over anhydrous MgSO₄, filter, and concentrate under reduce pressure. Purify the residue by flash chromatography eluting with NH₃ 7N in MeOH: DCM (0-10% gradient). Concentrate appropriate fractions to afford rac-1-[(2R)-2-[[4-[[6-(1-hydroxyethyl)-3-isopropyl-imidazo[1,2-a]pyridin-8-yl]amino]-1-piperidyl]methyl]morpholin-4-yl]prop-2-en-1-one (chiral at asterisk). Following this preparation gave 72 mg (47% yield) as a yellow oil. ES/MS (m/z): 456 (M+H).

### Examples 4 & 5

Isomer 3: 1-[(2R)-2-[[4-[[6-(1-hydroxyethyl)-3-isopropyl-imidazo[1,2-a]pyridin-8-yl]amino]-1 -piperidyl]methyl]morpholin-4-yl]prop-2-en-1 -one,

Isomer 4: 1-[(2R)-2-[[4-[[6-(1-hydroxyethyl)-3-isopropyl-imidazo[1,2-a]pyridin-8-yl]amino]-1 -piperidyl]methyl]morpholin-4-yl]prop-2-en-1 -one

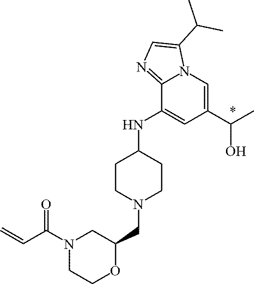

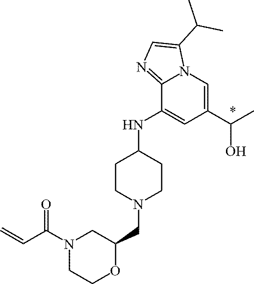

Purify racemic-1-[(2R)-2-[[4-[[6-(1-hydroxyethyl)-3-isopropyl-imidazo[1,2-a]pyridin-8-yl]amino]-1-piperidyl]methyl]morpholin-4-yl]prop-2-en-1-one (0.072 g, 0.141 mmol) by chiral chromatography. [Instrument: SFC10 (Sepiatec), Column: Chiralpak AD (25 cm x 2 cm, 5 um). Mobile phase: CO₂(A)/MeOH (0.2% DMEA)(B). Elution program: Isocratic 20%. Flow 65 mL/min. Loading: 15 mg injection every 9.92 min.] to afford both separated enantiomers:

Isomer 3: Following this method, 18.4 mg of 1-[(2R)-2-[[4-[[6-(1-hydroxyethyl)-3-isopropyl-imidazo[1,2-a]pyridin-8-yl]amino]-1-piperidyl]methyl]morpholin-4-yl]prop-2-en-1-one (27% yield) was obtained as a pale yellow oil. (Achiral purity by RP-LC/MS , Rt = 0.87 min,. 96%). (Chiral analysis, Rt = 0.92 min, e.e. >98%). ES/MS (m/z): 456 (M+H). ¹H NMR (400.21 MHz, DMSO): 7.47 (s, 1H), 7.15 (s, 1H), 6.82-6.75 (m, 1H), 6.15-6.11 (m, 2H), 5.71 (d, J= 10.3 Hz, 1H), 5.47-5.39 (m, 1H), 5.14 (d, J= 4.4 Hz, 1H), 4.73-4.67 (m, 1H), 4.41-4.23 (m, 1H), 4.00-3.86 (m, 2H), 3.51-3.47 (m, 3H), 3.25-3.20 (m, 1H), 3.01-2.92 (m, 3H), 2.48-2.33 (m, 3H), 2.30-2.17 (m, 2H), 1.95-1.92 (m, 2H), 1.58-1.50 (m, 2H), 1.38-1.29 (m, 9H).

Isomer 4: Following this method, 24.5 mg of 1-[(2R)-2-[[4-[[6-(1-hydroxyethyl)-3-isopropyl-imidazo[1,2-a]pyridin-8-yl]amino]-1-piperidyl]methyl]morpholin-4-yl]prop-2-en-1-one (36.7% yield) was obtained as a pale yellow oil. (Achiral purity by RP-LC/MS , Rt = 0.87 min,. 97%). (Chiral analysis, Rt = 1.16 min, e.e. >90%). ES/MS (m/z): 456(M+H). ¹H NMR (400.21 MHz, DMSO): 7.47 (s, 1H), 7.15 (s, 1H), 6.79 (dd, J= 10.4, 16.5 Hz, 1H), 6.15-6.11 (m, 2H), 5.71 (d, J= 10.3 Hz, 1H), 5.44-5.39 (m, 1H), 5.15 (d, J= 4.4 Hz, 1H), 4.73-4.67 (m, 1H), 4.41-4.14 (m, 1H), 3.97-3.80 (m, 3H), 3.23-3.13 (m, 2H), 2.98-2.85 (m, 3H), 2.45-2.33 (m, 3H), 2.26-2.14 (m, 2H), 1.95-1.92 (m, 2H), 1.60-1.47 (m, 2H), 1.38-1.29 (m, 9H).

While the Isomer 3 and Isomer 4 enantiomers of Examples 4 & 5 were separated, the specific chirality of each enantiomer at the asterisk position was not determined.

### Example 6

2,3,3-trideuterio-1-[(2R)-2-[[4-[[3-isopropyl-6-(trifluoromethyl)imidazo[1,2-a]pyridin-8-yl]amino]-1 -piperidyl]methyl]morpholin-4-yl]prop-2-en-1 -one

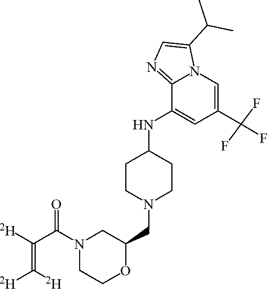 [SMILES: [2H]C([2H])=C([2H])C(=O)N1CCO[C@H](CN2CCC(Nc3cc(C(F)(F)F)cn4c(C(C)C)cnc34)CC2)C1]

Add 2,4,6-Tripropyl-1,3,5,2,4,6-trioxatriphosphorane-2,4,6-trioxide (50% in DMF) (0.25 mL, 0.42 mmol) to a solution of 2,3,3-trideuterioprop-2-enoic acid (0.029 g, 0.3863 mmol), prepared as described in Adv. Synth. Catal. 2018, 360, 2303, and 3-isopropyl-N-[1-[[(2S)-morpholin-2-yl]methyl]-4-piperidyl]-6-(trifluoromethyl)imidazo[1,2-a]pyridin-8-amine (0.15 g, 0.35 mmol) in dry DMF (3 mL) and TEA (0.2 mL, 1 mmol) at RT and stir the mixture for 2 hours. Quench the reaction mixture with 1 mL of a saturated aqueous solution of NaHCO₃ and extract with MTBE (twice). Separate and combine organic phases and dry over anhydrous Na₂SO₄. Filter and concentrate under reduced pressure. Purify the crude material by eluting with DCM: (DCM: MeOH 9/1) (0% isocratic), then gradient DCM: (DCM: MeOH 9/1) (0-60% gradient). Concentrate under reduced pressure and dry under high vacuum to afford 2,3,3-trideuterio-1-[(2R)-2-[[4-[[3-isopropyl-6-(trifluoromethyl)imidazo[1,2-a]pyridin-8-yl]amino]-1-piperidyl]methyl]morpholin-4-yl]prop-2-en-1-one. Following this preparation gave 85.6 mg (50% yield) as a white solid. ES/MS (m/z): 482 (M+H). (Achiral Rt= 1.128 min., 100%). ¹H NMR (400.21 MHz, DMSO): 8.02 (s, 1H, NH), 7.34 (s,1H), 6.11-6.03 (m, 1H), 4.40-4.12 (m, 1H), 4.00-3.80 (m, 2H), 3.51-3.27 (m, 3H), 3.22-3.08 (m, 1H), 2.90-2.80 (m, 3H), 2.41-2.40 (d, J= 5.5 Hz, 2H), 2.25-2.14 (m, 2H), 1.91-1.88 (m, 2H), 1.63-1.55 (m, 2H), 1.30 (d, J= 6 Hz, 6H).

### Preparation 27

8-chloro-3-[rac-1,2-dideuterio-1-methyl-ethyl]-6-(trifluoromethyl)imidazo[1,2-a]pyridine

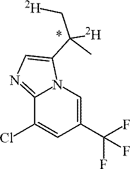

In the glove box, equally divide 1,1′-bis(di-I-propylphosphino)ferrocene(1,5-cyclooctadiene)rhodium(I) tetrafluoroborate (0.124 g, 0.17 mmol) between three 10-20 mL Biotage tubes with a stir bar. Dissolve 8-chloro-3-isopropenyl-6-(trifluoromethyl)imidazo[1,2-a]pyridine (1.84 g, 7.04 mmol) in THF (24 ml), and divide the solution evenly between the three vials. Cap the vials and remove from the glove box. Place the vials into an autoclave (together). Insert a needle into each vial to allow gas flow. Seal the autoclave and purge three times with deuterium to a final pressure of 80 psi. Stir the mixture for 3 h 40 min. Vent the autoclave and open the three tubes containing an orange solution. Rinse into a flask with EtOAc. Combine the three tubes and remove the solvent under reduced pressure. Dissolve the residue in DCM and purify by silica gel with a gradient of EtOAc: DCM (0-20% gradient). Remove solvent under reduced pressure to afford 8-chloro-3-[rac-1,2-dideuterio-1-methyl-ethyl]-6-(trifluoromethyl)imidazo[1,2-a]pyridine (chiral at asterisk). Following this preparation gave 1.72 g (92% yield) as a light yellow solid. ES/MS m/z (³⁵Cl/³⁷Cl): 265/267. ¹H NMR (399.80 MHz, DMSO): 8.93-8.92 (m, 1H), 7.75 (d, J= 1.5 Hz, 1H), 7.63 (s, 1H), 1.31-1.30 (m, 5H).

### Preparation 28

tert-butyl 4-[[3-[rac-1,2-dideuterio-1-methyl-ethyl]-6-(trifluoromethyl)imidazo[1,2-a]pyridin-8-yl]amino]piperidine-1-carboxylate

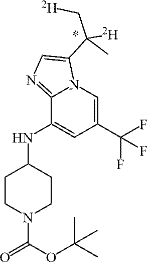

Add to a sealed tube 8-chloro-3-[rac-1,2-dideuterio-1-methyl-ethyl]-6-(trifluoromethyl)imidazo[1,2-a]pyridine (0.503 g, 1.90 mmol,), tert-butyl 4-aminopiperidine-1-carboxylate (0.418 g, 2.09 mmol), sodium tert-butoxide (0.6 g, 6 mmol), and 1,4-dioxane (10 mL). Purge the mixture with N₂ followed by vacuum (3 cycles). Add brettphos Pd G3 (0.1 g, 0.1 mmol), purge again with N₂-vacuum, cap the tube and heat the mixture at 95° C. for 2.5 h. Cool down the reaction mixture to RT, dilute the mixture with MTBE and wash with water. Separate organic phase and extract aqueous layer with MTBE (3X). Combine organic phases, dry over anhydrous Na₂SO₄, filter and concentrate under reduced pressure to afford a brown oil residue. Purify the residue by silica gel cartridge eluting with DCM/Hex and DCM/MeOH (1:1) (0-2% gradient). Collect appropriate fractions, remove volatiles and dry under high vacuum to afford tert-butyl 4-[[3-[rac-1,2-dideuterio-1-methyl-ethyl]-6-(trifluoromethyl)imidazo[1,2-a]pyridin-8-yl]amino]piperidine-1-carboxylate (chiral at asterisk). Following this preparation gave 0.52 g (55% yield) as a yellow foam. ES/MS (m/z): 429 (M+H).

### Preparation 29

3-(rac-1,2-dideuterio-1-methyl-ethyl)-N-(4-piperidyl)-6-(trifluoromethyl)imidazo[1,2-a]pyridin-8-amine

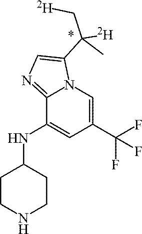

Add TFA (1.6 mL, 21 mmol) to a solution of tert-butyl 4-[[3-[rac-1,2-dideuterio-1-methyl-ethyl]-6-(trifluoromethyl)imidazo[1,2-a]pyridin-8-yl]amino]piperidine-1-carboxylate (0.524 g, 1.05 mmol) in DCM (7 mL) and stir the mixture at RT for 3 h. Treat the reaction mixture with water (20 mL), separate the organic layer, and discard. Treat aqueous layer with a saturated solution of NaHCO₃ and extract with DCM (3× 20 mL). Combine organic phases, dry over anhydrous Na₂SO₄, filter, and concentrate to yield 3-(rac-1,2-dideuterio-1-methyl-ethyl)-N-(4-piperidyl)-6-(trifluoromethyl)imidazo[1,2-a]pyridin-8-amine (chiral at asterisk). Following this preparation gave 0.27 g (78% yield) as a yellow solid. ES/MS (m/z) 329 (M+H)⁺. ¹H NMR (400.21 MHz, CDCl₃): 7.68 (d, J= 1.2 Hz, 1H), 7.29 (d, J= 7.1 Hz, 1H), 6.15 (d, J= 1.2 Hz, 1H), 5.36-5.32 (m, 1H), 3.62-3.53 (m, 1H), 3.22 (d, J= 11.7 Hz, 2H), 2.83 (t, J= 10.0 Hz, 2H), 2.25-2.04 (m, 3H), 1.58-1.50 (m, 2H), 1.40-1.39 (m, 5H).

### Preparation 30

tert-butyl (2R)-2-[[4-[[3-[rac-1,2-dideuterio-1-methyl-ethyl]-6-(trifluoromethyl)imidazo[1,2-a]pyridin-8-yl]amino-piperidyl]methyl]morpholine-4-carboxylate

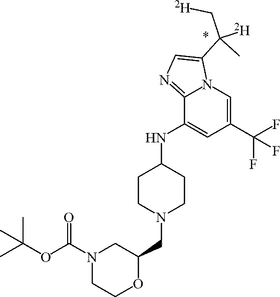

Add to a sealed tube 3-(rac-1,2-dideuterio-1-methyl-ethyl)-N-(4-piperidyl)-6-(trifluoromethyl)imidazo[1,2-a]pyridin-8-amine (0.27 g, 0.82 mmol), tert-butyl (2S)-2-(p-tolylsulfonyloxymethyl)morpholine-4-carboxylate (0.361 g, 0.97 mmol), and ACN (4 mL). Add TEA (0.23 mL, 1.7 mmol) to the resulting orange solution. Cap the flask and heat the mixture at 95° C. for 24 h. Remove the volatiles under vacuo and treat the residue with DCM (20 mL) and water (20 mL). Separate the organic layer and extract the aqueous layer with DCM (2×). Dry combined organics over anhydrous Na₂SO₄, filter, and concentrate under reduced pressure. Purify the residue by silica gel eluting with MeOH: DCM (0-3% gradient). Combine appropriate fractions, remove volatiles and dry under high vacuum to afford tert-butyl (2R)-2-[[4-[[3-[rac-1,2-dideuterio-1-methylethyl]-6-(trifluoromethyl)imidazo[1,2-a]pyridin-8-yl]amino]-1-piperidyl]methyl]morpholine-4-carboxylate (chiral at asterisk). Following this preparation gave 0.34 g (69% yield) as a reddish solid. ES/MS (m/z): 528 (M+H).

### Preparation 31

3-[rac-1,2-dideuterio-1-methyl-ethyl]-N-[1-[[(2S)-morpholin-2-yl]methyl]-4-piperidyl]-6-(trifluoromethyl)imidazo[1,2-a]pyridin-8-amine

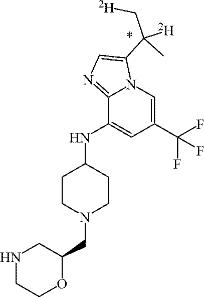

Add TFA (0.855 mL, 11.3 mmol) to a solution of tert-butyl (2R)-2-[[4-[[3-[rac-1,2-dideuterio-1-methyl-ethyl]-6-(trifluoromethyl)imidazo[1,2-a]pyridin-8-yl]amino]-1-piperidyl]methyl]morpholine-4-carboxylate (338 mg, 0.56 mmol) in DCM (5 mL). Stir the resulting mixture at RT for 16 h. Treat the reaction mixture with saturated aqueous NaHCO₃ (30 mL) and extract with DCM (2 x 15 mL). Combine organic phases, dry over anhydrous Na₂SO₄, filter, and concentrate under reduced pressure to yield 3-[rac-1,2-dideuterio-1-methyl-ethyl]-N-[1-[[(2S)-morpholin-2-yl]methyl]-4-piperidyl]-6-(trifluoromethyl)imidazo[1,2-a]pyridin-8-amine (chiral at asterisk). Following this preparation gave 222 mg (82.3% yield) as a reddish foam. ES/MS (m/z): 428 (M+H).

### Example 7

1-[(2R)[[4-[[3-[rac-1,2-dideuterio-1-methyl-ethyl]-6-(trifluoromethyl)imidazo[1,2-a]pyridin-8-yl]amino]-1-piperidyl]methyl]morpholin-4-yl]propen-1-one

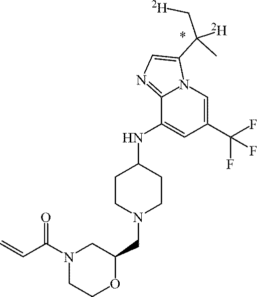

Add TEA (0.199 mL, 1.43 mmol) followed by acryloyl chloride (0.048 mL, 0.58 mmol) to a solution of 3-[rac-1,2-dideuterio-1-methyl-ethyl]-N-[1-[[(2S)-morpholin-2-yl]methyl]-4-piperidyl]-6-(trifluoromethyl)imidazo[1,2-a]pyridin-8-amine (220 mg, 0.46 mmol) in dichloromethane (2.2 mL) cooled at 0° C. Stir the resulting mixture at this temperature for 20 min. Quench the reaction mixture with 5 mL of saturated aqueous NaHCO₃ and extract with DCM (2x). Combine the organic layers, dry over anhydrous Na₂SO₄, filter, and concentrate under reduced pressure. Purify the residue by silica gel eluting with MeOH: DCM (0- 4% gradient). Combine appropriate fractions, remove volatiles, and dry under high vacuum to afford 1-[(2R)-2-[[4-[[3-[rac-1,2-dideuterio-1-methyl-ethyl]-6-(trifluoromethyl)imidazo[1,2-a]pyridin-8-yl]amino]-1-piperidyl]methyl]morpholin-4-yl]prop-2-en-1-one (chiral at asterisk). Following this preparation gave 103 mg (45.3% yield) as a reddish foam. ES/MS (m/z): 482 (M+H). ¹H NMR (400.21 MHz, CD₃OD): 7.96 (s, 1H), 7.34 (s, 1H), 6.83-6.73 (m, 1H), 6.30-6.22 (m, 2H), 5.79 (d, J= 10.5 Hz, 1H), 4.48-4.45 (m, 4H), 4.07-3.96 (m, 2H), 3.68-3.56 (m, 3H), 3.15-2.91 (m, 2H), 2.67-2.49 (m, 4H), 2.13 (dd, J= 4.0, 8.9 Hz, 2H), 1.75-1.67 (m, 2H), 1.40-1.38 (m, 5H).

### Preparation 33

tert-butyl (2S)-2-(p-tolylsulfonyloxymethyl)morpholine-4-carboxylate

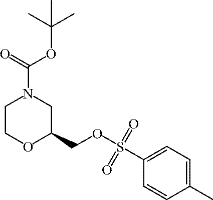 [SMILES: Cc1ccc(S(=O)(=O)OC[C@@H]2CN(C(=O)OC(C)(C)C)CCO2)cc1]

Add DMAP (5.62 g, 46.1 mmol, 0.1 eq), TEA (112 g, 154 mL, 1.11 mol, 2.4 eq), and p-toluenesulfonyl chloride (105.4 g, 553 mmol, 1.2 eq) to a solution of tert-butyl (2S)-2-(hydroxymethyl)morpholine-4-carboxylate (100 g, 461 mmol) in THF (921 mL), and stir the mixture for 24 h at 23° C. Add 9% aqueous NaHCO₃ (1151 mL) and extract with EtOAc (506 mL). Wash the organic phase with saturated aqueous NaCl (506 mL), dry over anhydrous MgSO₄, filter, and concentrate in vacuo. Add heptane (1000 mL) to the residue, and stir for 24 h. Filter, wash with heptane (2 × 150 mL), and dry under a stream of air for 1 h and under 10 mbar vacuum, 45° C. overnight to obtain tert-butyl (2S)-2-(p-tolylsulfonyloxymethyl)morpholine-4-carboxylate. Following this preparation gave 147 g (86% yield) as a white solid. ES/MS m/z 394 (M+Na)⁺.

### Preparation 34

tert-butyl (2R)-2-[(4-amino-1-piperidyl)methyl]morpholine-4-carboxylate

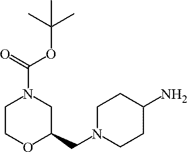 [SMILES: CC(C)(C)OC(=O)N1CCO[C@H](CN2CCC(N)CC2)C1]

Charge a pressure vessel with tert-butyl (2S)-2-(p-tolylsulfonyloxymethyl)morpholine-4-carboxylate (81.2 g, 216 mmol), piperidin-4-amine (43.3 g, 433 mmol, 2 eq), methyl ethyl ketone (216 mL), and DIPEA (55.9 g, 75.5 mL, 433 mmol, 2 eq). Stir the mixture at 80° C. for 40 h. Turn the heating off, cool down to 23° C., dilute with MTBE (649 mL), wash with water (649 mL), 5% aqueous citric acid (649 mL), and back-extract the aqueous phase with MTBE (649 mL). Basify the combined aqueous phases by stirring with a solution of 18 M aqueous NaOH (35.3 mL) and extract with DCM (3 × 649 mL). Dry the combined organics over anhydrous Na₂SO₄, filter, and concentrate in vacuo. Suspend the residue in heptane (649 mL) for 2 h, filter, concentrate in vacuo, and dry under 10 mBar at 45° C. for 48 h to obtain tert-butyl (2R)-2-[(4-amino-1-piperidyl)methyl]morpholine-4-carboxylate. Following this preparation gave 47.7 g (69% yield) as a colourless oil. ES/MS m/z 300 (M+H)⁺. ¹H NMR (CDCl₃) δ 1.34-1.62 (m, 2H), 1.47 (s, 9H), 1.71-1.85 (m, 2H), 2.00-2.18 (m, 2H), 2.20-2.38 (m, 1H), 2.50 (dd, 1H), 2.54-2.74 (m, 2H), 2.83-2.99 (m, 2H), 3.44-3.63 (m, 2H), 3.78-4.04 (m, 4H).

### Preparation 35

8-chloro-3-iodo-6-(trifluoromethyl)imidazo[1,2-a]pyridine

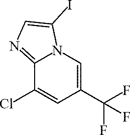 [SMILES: FC(F)(F)c1cc(Cl)c2ncc(I)n2c1]

Add NIS (69.5 g, 303 mmol, 1.3 eq) to a solution of 8-chloro-6-(trifluoromethyl)imidazo[1,2-a]pyridine (51.4 g, 233 mmol) in ACN (1164 mL), and stir the mixture at 23° C. for 72 h. Concentrate in vacuo. Dissolve the residue in 2-Me-THF (514 mL), and wash with 25% aqueous Na₂S₂O₃ (514 mL), and with 9% aqueous NaHCO₃ (3 × 514 mL). Dry the organic phase over anhydrous MgSO₄, filter, and concentrate in vacuo to obtain 8-chloro-3-iodo-6-(trifluoromethyl)imidazo[1,2-a]pyridine. Following this preparation gave 71.3 g (88% yield) as a white solid. ES/MS m/z (³⁵Cl/³⁷Cl) 346/348. ¹H NMR (CDCl₃) δ 7.53 (d, 1H), 7.89 (s, 1H), 8.46 (m, 1H).

### Preparation 36

8-chloro-3-isopropenyl-6-(trifluoromethyl)imidazo[1,2-a]pyridine

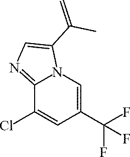 [SMILES: C=C(C)c1cnc2c(Cl)cc(C(F)(F)F)cn12]

Degas under N₂ a mixture of 8-chloro-3-iodo-6-(trifluoromethyl)imidazo[1,2-a]pyridine (25.1 g, 72.4 mmol), EtOH (483 mL), and 1.2 M aqueous K₂CO₃ (180 mL) for 5 min. Add 2-isopropenyl-4,4,5,5-tetramethyl-1,3,2-dioxaborolane (14.1 g, 79.6 mmol, 1.1 eq) and BrettPhos Pd G3 (1.67 g, 1.81 mmol, 0.025 eq), and degas for 5 min. Stir at 23° C. for 24 h. Concentrate in vacuo, dissolve the residue in 2-MeTHF (251 mL), and wash with water (125 mL). Dry the organic phase over anhydrous MgSO₄, filter, and concentrate in vacuo. Purify the residue by silica gel chromatography EtOAc: hexane (0-50% gradient), to obtain 8-chloro-3-isopropenyl-6-(trifluoromethyl)imidazo[1,2-a]pyridine. Following this preparation gave 16.9 g (85% yield) as a pale yellow solid. ES/MS m/z (³⁵Cl/³⁷Cl) 261/263. ¹H NMR (d6-DMSO) δ 2.22 (m, 3H), 5.47 (m, 1H), 5.51 (m, 1H), 7.70 (d, 1H), 7.86 (s, 1H), 8.61 (m, 1H).

### Preparation 37

8-chloro-3-isopropyl-6-(trifluoromethyl)imidazo[1,2-a]pyridine

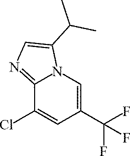 [SMILES: CC(C)c1cnc2c(Cl)cc(C(F)(F)F)cn12]

Degas under N₂ a mixture of 8-chloro-3-isopropenyl-6-(trifluoromethyl)imidazo[1,2-a]pyridine (33.2 g, 110 mmol), platinum (20.1 g, 2.31 mmol, 0.021 eq), and MeOH (659 mL). Replace the N₂ atmosphere with H₂ using three vacuum cycles. Stir the mixture at 23° C. for 8 h. Filter through a pad of diatomaceous earth and rinse with MeOH. Concentrate in vacuo. Purify the residue by silica gel chromatography EtOAc: hexane (10-70% gradient) to obtain 8-chloro-3-isopropyl-6-(trifluoromethyl)imidazo[1,2-a]pyridine. Following this preparation gave 24.2 g (65% yield) as a yellow solid ES/MS m/z (³⁵Cl/³⁷Cl) 263/265. ¹H NMR (d₆-DMSO) δ 1.33 (d, 6H), 3.51 (dq, 1H), 7.63 (d, 1H), 7.75 (d, 1H), 8.92 (m, 1H).

### Preparation 38

tert-butyl (2R)-2-[[4-[[3-isopropyl-6-(trifluoromethyl)imidazo[1,2-a]pyridin-8-yl]amino]-1-piperidyl]methyl]morpholine-4-carboxylate

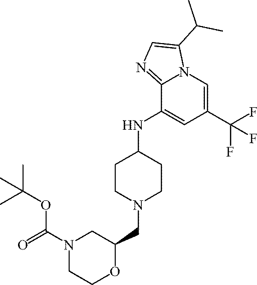 [SMILES: CC(C)c1cnc2c(NC3CCN(C[C@@H]4CN(C(=O)OC(C)(C)C)CCO4)CC3)cc(C(F)(F)F)cn12]

Degas under N₂ a mixture of 8-chloro-3-isopropyl-6-(trifluoromethyl)imidazo[1,2-a]pyridine (22.7 g, 79.2 mmol), tert-butyl (2R)-2-[(4-amino-1-piperidyl)methyl]morpholine-4-carboxylate (37.9 g, 119 mmol, 1.5 mmol), sodium tert-butoxide (23.5 g, 238 mmol, 3 eq), and 1,4-dioxane (396 mL). Add BrettPhos Pd G3 (4.53 g, 4.75 mmol, 0.06 eq) and degas the mixture for 5 min. Heat at 100° C. for 48 h. Cool to 23° C., concentrate in vacuo, dissolve the residue in 2-MeTHF (227 mL), and wash with water (127 mL). Extract the aqueous phase with 2-MeTHF (114 mL), dry the combined organics over anhydrous MgSO₄, filter, and concentrate in vacuo to obtain tert-butyl (2R)-2-[[4-[[3-isopropyl-6-(trifluoromethyl)imidazo[1,2-a]pyridin-8-yl]amino]-1-piperidyl]methyl]morpholine-4-carboxylate. Following this preparation gave 59.7 g (70% pure, 100% yield) as a brown gum. ES/MS m/z 526 (M+H)⁺. ¹H NMR (d₆-DMSO) δ 1.30 (d, 6H), 1.41 (s, 9H), 1.52-1.68 (m, 2H), 1.81-1.95 (m, 2H), 2.09-2.29 (m, 2H), 2.31-2.43 (m, 2H), 2.75-2.94 (m, 3H), 3.25-3.58 (m, 5H), 3.62-3.92 (m, 3H), 6.07 (d, 1H), 6.21 (d, 1H), 7.34 (d, 1H), 8.03 (m, 1H).

### Preparation 39

3-isopropyl-N-[1-[[(2S)-morpholin-2-yl]methyl]-4-piperidyl]-6-(trifluoromethyl)imidazo[1,2-a]pyridin-8-amine

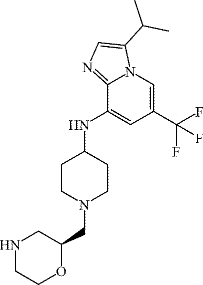 [SMILES: CC(C)c1cnc2c(NC3CCN(C[C@@H]4CNCCO4)CC3)cc(C(F)(F)F)cn12]

Add HCl in 2-propanol (5.5 M, 86 mL, 6 eq.) to a suspension of tert-butyl (2R)-2-[[4-[[3-isopropyl-6-(trifluoromethyl)imidazo[1,2-a]pyridin-8-yl]amino]-1-piperidyl]methyl]morpholine-4-carboxylate (59.5 g, 70% pure, 79.1 mmol) in 2-propanol (476 mL). Heat the mixture at 95° C. for 4 h. Cool to 23° C. and concentrate in vacuo. Suspend the residue in 2-MeTHF (476 mL), add a solution of 2 M aqueous NaOH (198 mL), and stir at 23° C. for 5 min. Filter through a pad of diatomaceous earth and rinse with 2-MeTHF. Extract the organic phase with 20% aqueous citric acid (2 × 476 mL). Treat the combined aqueous phases with 18.4 M aqueous NaOH (476 mL) and extract with 2-MeTHF (2 × 476 mL). Dry the combined organics over anhydrous MgSO₄, filter, and concentrate in vacuo. Treat the residue at 23° C. with SiliaMetS® Thiol resin (40-63 µm; loading = 1.46 mmol/g; 10.8 g, 15.7 mmol), and heat to 65° C. for 18 h. Cool to 23° C., filter, and concentrate in vacuo to obtain 3-isopropyl-N-[1-[[(2S)-morpholin-2-yl]methyl]-4-piperidyl]-6-(trifluoromethyl)imidazo[1,2-a]pyridin-8-amine. Following this preparation gave 27.2 g (76% yield) as an orange solid. ES/MS m/z 426 (M+H)⁺. ¹H NMR (d₆-DMSO) δ 1.30 (d, 6H), 1.51-1.68 (m, 2H), 1.80-1.94 (m, 2H), 2.07-2.23 (m, 2H), 2.23-2.42 (m, 3H), 2.58-2.76 (m, 2H), 2.76-2.92 (m, 3H), 3.28-3.59 (m, 5H), 3.66-3.78 (m, 1H), 6.04 (d, 1H), 6.21 (bs, 1H), 7.34 (bs, 1H), 8.02 (m, 1H).

### Example 8

1-[(2R)[[4-[[3-isopropyl-6-(trifluoromethyl)imidazo[1,2-a]pyridin-8-yl]amino]-1-piperidyl]methyl]morpholin-4-yl]propen-1-one

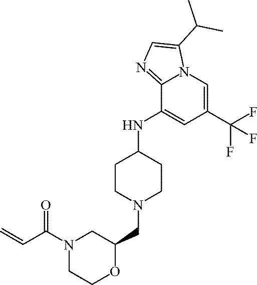 [SMILES: C=CC(=O)N1CCO[C@H](CN2CCC(Nc3cc(C(F)(F)F)cn4c(C(C)C)cnc34)CC2)C1]

Add dropwise TEA (16.3 g, 22.4 mL, 161 mmol, 4 eq), and a solution of acryloyl chloride (3.79 g, 3.40 mL, 40.1 mmol, 1.0 eq) in DCM (34 mL), to a solution of 3-isopropyl-N-[1-[[(2S)-morpholin-2-yl]methyl]-4-piperidyl]-6-(trifluoromethyl)imidazo[1,2-a]pyridin-8-amine (17.7 g, 40.1 mmol) in DCM (268 mL) at 0° C. and stir for 15 min. Add 9% aqueous NaHCO₃ (142 mL), dry the organic phase over anhydrous MgSO₄, filter, and concentrate in vacuo. Purify the residue by silica gel chromatography MeOH: DCM (0-10% gradient), to obtain 1-[(2R)-2-[[4-[[3-isopropyl-6-(trifluoromethyl)imidazo[1,2-a]pyridin-8-yl]amino]-1-piperidyl]methyl]morpholin-4-yl]prop-2-en-1-one. Following this preparation gave 12.9 g (67% yield) as a beige foam. ES/MS m/z 480 (M+H)⁺. ¹H NMR (d₆-DMSO) δ 1.30 (d, 6H), 1.52-1.71 (m, 2H), 1.83-1.95 (m, 2H), 2.10-2.30 (m, 2H), 2.36-2.46 (m, 2H), 2.74-3.23 (m, 3H), 3.31-3.60 (m, 5H), 3.78-4.02 (m, 2H), 4.08-4.47 (m, 1H), 5.71 (d, 1H), 6.06 (m, 1H), 6.14 (dd, 1H), 6.22 (d, 1H), 6.79 (dd, 1H), 7.39 (d, 1H), 8.02 (m, 1H).

### Biological Assays

The following assays demonstrate that the compounds described herein are inhibitors of CDK7 activity. The results of the assays also show that the compounds described herein inhibit CDK7 signaling in the cancer cells. Additionally, the compounds described herein inhibit proliferation in cancer cell lines and tumor growth in xenograft tumor model of cancer.

“IC₅₀” refers to the concentration of an agent that produces 50% of the maximal inhibitory response possible for that agent or, alternatively, to the concentration of an agent which produces 50% displacement of ligand specific binding to the receptor; Relative IC₅₀ values are determined using fluorescence unit by calculating percent inhibition with respect to on-plate “MIN” and “MAX” controls and then fitting the ten-point dose response data to a four-parameter logistic equation.

### CDK7 and CDK9 Kinase Activity Assays

The purpose of these assays is to measure the ability of the compounds described herein to inhibit CDK7/CyclinH/Mat1 complex kinase activity. To demonstrate whether the compounds described herein exhibit any affinity for CDK7 and CDK9, the biochemical assays are performed with no preincubation of the enzyme with the compound or with 3 hours preincubation. Functional assays provide support on whether the compounds described herein exhibit the ability to inhibit the CDK7 and CDK9 kinase activities. All ligands, solvents, and reagents employed in the following assays are readily available from commercial sources, or can be readily synthesized by one skilled in the art. The IC₅₀ determination for CDK7 and CDK9 are determined as follows.

### Biochemical Assay for Inhibition of CDK7/CyclinH/MATl

The IC₅₀ activity of the compounds described herein is determined using radiolabel filter binding (FB) assays using the purified human recombinant enzyme in the presence of ATP//[³³P]ATP and peptide substrate. The ATP concentrations chosen are at or near the enzyme Kₘ for ATP.

Reactions are carried out in 96 well polystyrene plates in a final volume of 25 µL per well. 5 µL of test compound in 20% DMSO, 10 µL of substrate solution (ATP/33PATP and CDK7/9 tide), and 10 µL of enzyme solution are mixed. The substrate solution is prepared to give a final concentration of 100 µM ATP/[³³P]ATP (NEN 10µCi/µL, 3000 Ci/mmol) and 250 µM CDK7/9 peptide ((YSPTSPSYSPTSPSYSPTSPSKKKK) (SEQ ID NO: 1)) diluted in kinase buffer of 4 mM MgCl2, 0.01% TRITON™ X-100, 2 mM DTT and 20 mM HEPES. The enzyme solution is prepared for a final concentration of 1 nM CDK7/CyclinH/Mat1 enzyme [Proqinase 0366-0360-4 Lot 002)] diluted in kinase buffer. Test compounds are serially diluted 1:3 in 20% DMSO to create a 10 point curve at a starting concentration of 20 µM. 20% DMSO buffer alone without test compound is employed as high control (full activity in the absence of any inhibitor), 500 mM EDTA is used to determine the level of background in the absence of enzyme activity (low control). After mixing 5 µL of compounds with 10 µL of enzyme solution, the plate is incubated for 0 or 180 minutes at 22° C. After that time, the reaction is initiated by the addition of 10 µL substrate solution and incubated for 50 minutes at 22° C. The reaction is terminated by the addition of 80 µL of cold 10% orthophosphoric solution. The Filter Plates (opaque, non-sterile filter plates) are prewashed with 10 µL of 10% orthophosphoric solution to each well. 100 µL of the mixture are transferred to a phosphocellulose filter and incubated at room temperature for 45 minutes. Filter plates are washed with 200 µL 0.5 % orthophosphoric acid 3 times on a filter plate processor. Incorporation of 33Pi (counting of “cpm”) is determined by adding 80 µL of MICROSCINT™ to each well and read on a counter after an hour. Data is processed through a GENEDATA SCREENERⓇ tool. Data are analyzed using a 4-parameter nonlinear logistic equation (four-parameter logistic concentration-response curve): Y = bot + [(top-bot)/1+(x/ IC₅₀)slope] where Y = % inhibition, X = concentration yielding y% inhibition, Bottom = minimum value of y attained by curve, Top = maximum value of y attained by curve and Slope = steepness of curve at IC₅₀. %Inh = [(median Max- x/ median Max - median Min)] . 100 IC₅₀: concentration of compound that reduces a given response (ligand binding, enzyme response) by 50%.

The compounds described in Examples 1, 2, 4, 5, 6, 7, and 8 display an IC₅₀ of 0.123 µM, 0.256 µM, 0.155 µM, 0.367 µM, 0.0674 µM, 0.0845 µM, and 0.0656 µm in CDK7 without preincubation, respectively. After 3 hours of preincubation of CDK7 enzyme with Examples 1, 2, 4, 5, 6, 7, and 8, they show an IC₅₀ of 0.0143 µM, 0.0266 µM, 0.0143 µM, 0.0415 µM, 0.00396 µM, 0.00625 µM, and 0.00574 µM, respectively. These data show that Examples 1, 2, 4, 5, 6, 7, and 8 inhibit CDK7.

### Assay for Inhibition of CDK9/CyclinTl Kinase Activity

The IC₅₀ activity of the compounds described herein is determined using radiolabel filter binding (FB) assays using the purified human recombinant enzyme in the presence of ATP and peptide substrate. The ATP concentrations chosen are at or near the enzyme Km for ATP. Reactions are carried out in 96 well polystyrene plates in a final volume of 25 µL per well. 5 µL of test compound in 20% DMSO, 10 µL of substrate solution (ATP//[³³P]ATP and CDK7/9 tide) and 10 µL of enzyme solution are mixed. The substrate solution is prepared to give a final concentration of 100 µM ATP/[³³P]ATP (NEN 10uCi/µL, 3000 Ci/mmol) and 200 µM CDK7/9 peptide ((YSPTSPSYSPTSPSYSPTSPSKKKK) (SEQ ID NO: 1)) diluted in kinase buffer of 4 mM MgCl2, 0.0025% TRITON™ X-100, 1.58 mM DTT, and 15.80 mM HEPES. The enzyme solution is prepared for a final concentration of 7.5 nM CDK9/cyclinTl enzyme [Proqinase 0371-0345-1 (Lot 004)] diluted in kinase buffer. Test compounds are serially diluted 1:3 in 20% DMSO to create a 10 point curve at a starting concentration of 20 µM. 20% DMSO buffer alone without test compound is employed as high control (full activity in the absence of any inhibitor), 500 mM EDTA is used to determine the level of background in the absence of enzyme activity (low control). After mixing 5 µL of compounds with 10 µL of enzyme solution, the plate is incubated for 0 or 180 minutes at 22° C. After that time, the reaction is initiated by the addition of 10 µL substrate solution and incubated for 60 minutes at 22° C. The reaction is terminated by the addition of 80 µL of cold 10% orthophosphoric solution. Filter plates (opaque, non-sterile filter plates) are prewashed with 10 µL of 10% orthophosphoric solution per well. 100 µL of the mixture are transferred to a phosphocellulose filter and incubate at room temperature for 45 minutes. Filter plates are washed with 200 µL 0.5 % orthophosphoric acid 3 times on a filter plate processor. 80 µL of MICROSCINT™ is added to each well and read on a scintillation counter after an hour. Data is processed through a GENEDATA-SCREENERⓇ tool. Data is analyzed using a 4-parameter nonlinear logistic equation (four-parameter logistic concentration-response curve): Y = bot + [(top-bot)/1+(x/ IC₅₀)_{S}lope] where Y = % inhibition, X = concentration yielding y% inhibition, Bottom = minimum value of y attained by curve, Top = maximum value of y attained by curve and Slope = steepness of curve at IC₅₀. %Inh = [(median Max- x/ median Max - median Min)] . 100 IC₅₀: concentration of compound that reduces a given response (ligand binding, enzyme response) by 50%. IC₅₀ relative: concentration giving half the compound’s maximum response.

The compounds described in Examples 1, 2, 4, 5, 6, 7, and 8 display an IC₅₀ of 1.77 µM, 3.18 µM, 8.05 µM, 7.13 µM, 1.61 µM, 2.03 µM, and 2.14 µM for CDK9 (3 hours preincubation), respectively. These data show that Examples 1, 2, 4, 5, 6, 7, and 8 do not potently inhibit CDK9 activity.

Taken together, the data from the assays above demonstrate that the compounds of Examples 1, 2, 4, 5, 6, 7, and 8 selectively inhibit CDK7 over CDK9.

### CDK7 and CDK9 Cell Mechanistic Assays

The purpose of these assays is to measure the ability of the compounds described herein to inhibit CDK7 and CDK9 signaling in cancer cells in vitro.

### Phospho-Carboxyl Terminal Domain (Rbp2) (Ser2) p-CTD (S2) Cell Based Acumen Assay

HCT116 cells (ATCC CCL-247) are cultured in McCoy’s 5 Å Medium Modified media supplemented with 10% FBS, 1% NaPyr, and 1% Pen/Strep and plated (prior to becoming 70% confluent) in 96-well flat-bottom plates at a density of 5,000 cells per well in 100 µL volume. The cells are then incubated overnight in a cell culture incubator (5% CO₂, 95% Relative Humidity (RH) and 37° C.) and allowed to attach to the plate. The following morning the cells are dosed with compounds. Compound inhibitors are first solubilized at 60 µM in culture medium containing 0.6% DMSO. Subsequently compound serial dilutions (1:3) are prepared over a 60 µM to 0.003 µM range. Cells are dosed with the addition of 50 µL from serial dilution plate to assay plate containing cells attached with 100 µL of media producing a final DMSO concentration of 0.2% with a final compound concentration dose range between 20 and 0.001 µM. For max point media containing 0.2% of DMSO is used and for min point, a reference compound diluted at 0.83 µM final concentration in the growth media containing 0.2% DMSO is used. After dosing with compounds, the cell plates are incubated at 37° C. and 5% CO₂ for 4 hours. The growth media is removed carefully and the cells are fixed by adding 100 µL of 4% para-formaldehyde for 30 minutes at RT. Cells are washed once with PBS and incubated with 100 µL of cold MeOH for 15 minutes at RT for cell permeation. Cells are washed twice with PBS (100 µL/each) and blocked with 100 µL/well of 1% BSA/PBS for 30 minutes at RT. 50 µL of 1:1000 primary antibody (Anti-phospho CTD Ser2 Abcam, cat# ab5095-100) dilution in 1% BSA/PBS are added per well, the plates are sealed and incubated overnight at 4° C.

The following day cells are washed three times with PBS (100 µL/well) and incubated with 50 µL/well of secondary antibody (1:2000 dilution, Goat anti-rabbit IgM ALEXA FLUOR™ 488) in PBS for 1 hour at RT. After washing 3X with PBS (100 µL/well), 100 µL of 50 µg/mL RNAase and 1:1000 propidium iodide dilution in PBS are added per well. Plates are sealed and incubated 1 hour at RT on the bench (preserved from light). Plates are analyzed on Acumen on FL2 (mean intensity) and FL3 (total intensity). Fluorescence plates are scanned with ACUMEN EXPLORER™ [Laser-scanning fluorescence microplate cytometer manufactured by TTP LABTECH LTD] to measure anti-phospho-carboxyl terminal domain at Serine 2 (pCTD). Image analysis is based on cellular fluorescent signals for identifying positive cells. pCTD (S2) positive cells are identified by mean intensity at 500-530 above the threshold. Total intensity at 575-640 from propidium iodide/DNA is used to identify individual cells. Assay output is % pCTD positive cells.

The IC₅₀ is determined by curve fitting to a four parameter logistic for each output using GENE DATA™. The compounds described in Examples 1, 2, 4, 5, 6, 7, and 8 display a relative IC₅₀ of 5.73 µM, 6.36 µM, 3.71 µM, 7.79 µM, 3.79 µM, 2.92 µM, and 2.59 µM for phosphoCTD (S2), respectively. These data show that Examples 1, 2, 4, 5, 6, 7, and 8 do not potently inhibit CDK9 in the cells.

### Phospho-Carboxyl Terminal Domain (Rbp2) (Ser5) p-CTD (S5) Cell Based Acumen Assay

HCT116 cells (ATCC CCL-247) are cultured in McCoy’s 5A Medium Modified media supplemented with 10% FBS, 1% NaPyr, and 1% Pen/Strep and plated (prior to becoming 70% confluent) in 96-well flat-bottom plates at a density of 5,000 cells per well in 100 µL volume. The cells are incubated overnight in a cell culture incubator (5% CO₂, 95% Relative Humidity (RH), and 37° C.) and allowed to attach to the plate. The following morning, the cells are dosed with compounds. Compound inhibitors are solubilized at 60 µM in culture medium containing 0.6% DMSO. Subsequently compound serial dilutions (1:3) are prepared over a 60 µM to 0.003 µM range. Cells are dosed with the addition of 50 µL from serial dilution plate to assay plate containing cell attached with 100 µL of media producing a final DMSO concentration of 0.2% with a final compound concentration dose range between 20 and 0.001 µM. For max point media containing 0.2% of DMSO is used and for min point, a reference compound diluted at 0.83 µM final concentration in the growth media containing 0.2% DMSO is used. After dosing with compounds, the cell plates are incubated at 37° C. and 5% CO₂ for 4 hours. Growth media is removed carefully and the cells are fixed by adding 100 µL of 4% para-formaldehyde for 30 minutes at RT. Cell are washed once with PBS and incubated with 100 µL of cold MeOH for 15 minutes at RT for cell permeation. Again cells are washed twice with PBS (100 µL/each) and blocked with 100 µL/well of 1% BSA/PBS for 30 min at RT. 50 µL of 1:1000 primary antibody (Anti-phosphoCTD Ser5 Bethyl Laboratories cat# A300-655A) dilution in 1% BSA/PBS are added per well, the plates are sealed and incubated overnight at 4° C.

The following day cells are washed three times with PBS (100 µL/well) and incubated with 50 µL/well of secondary antibody (1:2000 dilution, Goat anti-rabbit IgM ALEXA FLUOR™ 488) in PBS for 1 hourr at room temperature. After washing 3× with PBS (100 µL/well), 100 µL, of 50 µg/mL RNAase (Sigma) and 1:1000 propidium iodide dilution in PBS are added per well. Plates are sealed and incubated for 1 hour at RT on the bench (preserved from light). Plates are analyzed on Acumen on FL2 (mean intensity), and FL3 (total intensity). Fluorescence plates are scanned with ACUMEN EXPLORER™ [Laser-scanning fluorescence microplate cytometer manufactured by TTP LABTECH LTD] to measure anti-phospho-carboxyl terminal domain at Serine 5 (pCTD). Image analysis is based on cellular fluorescent signals for identifying positive cells. pCTD (S5) positive cells are identified by mean intensity at 500-530 above the threshold. Total intensity at 575-640 from propidium iodide/DNA is used to identify individual cells. Assay output is % pCTD positive cells. The IC₅₀ is determined by curve fitting to a four parameter logistic for each output using GENE DATA™.

The compounds described in Examples 1, 2, 4, 5, 6, 7, and 8 display a Relative IC₅₀ of 0.161 µM, 0.162 µM, 0.0551 µM, 0.118 µM, 0.0159 µM, 0.0717 µM, and 0.0262 µM for pCTD Ser5, respectively. These data show that Examples 1, 2, 4, 5, 6, 7, and 8 inhibit CDK7 cellular activity.

### cMyc Cell Based Acumen Assay

HCT116 cells (ATCC CCL-247) are cultured in McCoy’s 5 Å Medium Modified media supplemented with 10% FBS, 1% NaPyr, and 1% Pen/Strep and plated (prior to becoming 70% confluent) in 96-well flat-bottom plates at a density of 5,000 cells per well in 100 µL volume. The cells are then incubated overnight in a cell culture incubator (5% CO₂, 95% Relative Humidity (RH), and 37° C.) and allowed to attach to the plate. The following morning the cells are dosed with compounds. Compound inhibitors are solubilized at 60 µM in culture medium containing 0.6% DMSO. Subsequently, compound serial dilutions (1:3) are prepared over a 60 µM to 0.003 µM range. Cells are dosed with the addition of 50 µL from serial dilution plate to assay plate containing cell attached with 100 µL of media producing a final DMSO concentration of 0.2% with a final compound concentration dose range between 20 µM and 0.001 µM. For max point media containing 0.2% of DMSO is used and for min point, a reference compound diluted at 0.83 µM final concentration in the growth media containing 0.2% DMSO is used. After dosing with compounds, the cell plates are incubated at 37° C. and 5% CO₂ for 4 hours. Growth media is removed carefully and the cells are fixed by adding 100 µL of 4% para-formaldehyde for 30 minutes at RT. Cell are washed once with PBS and incubated with 100 µL of cold MeOH for 15 minutes at RT for cell permeation. Again cell are washed twice with PBS (100 µL/each) and blocked with 100 µL/well of 1% BSA/PBS for 30 minutes at RT. 50 µL of 1:1000 primary antibody (Anti-c-Myc antibody [Y69] Abcam cat# ab32072) dilution in 1% BSA/PBS are added per well, the plates sealed and incubated overnight at 4° C. The following day cells are washed three times with PBS (100 µL/well) and incubated with 50 µL/well of secondary antibody (1:2000 dilution, Goat anti-rabbit IgM ALEXA FLUOR™ 488) in PBS for 1 hour at RT. After wash 3× with PBS (100 µL/well), 100 µL of 50 µg/mL RNAase and 1:1000 propidium iodide (Invitrogene) dilution in PBS are added per well. Plates are sealed and incubated for 1 hour at RT on the bench (preserved from light). Plates are analyzed on Acumen on FL2 (mean intensity), and FL3 (total intensity). Fluorescence Plates are scanned with ACUMEN EXPLORER™ [Laser-scanning fluorescence microplate cytometer manufactured by TTP LABTECH LTD] to measure anti-phospho-carboxyl terminal domain at Serine 5 (pCTD). Image analysis is based on cellular fluorescent signals for identifying positive cells. pCTD (S5) positive cells are identified by mean intensity at 500-530 above the threshold. Total intensity at 575-640 from propidium iodide/DNA is used to identify individual cells. Assay output is % pCTD positive cells. The IC₅₀ is determined by curve fitting to a four parameter logistic for each output using GENE DATA™.

The compounds described in Examples 1, 2, 4, 5, 6, 7, and 8 display a Relative IC₅₀ of 0.082 µM, 0.0947 µM, 0.038 µM, 0.14 µM, 0.00791 µM, 0.0138 µM, and 0.0245 µM for cMyc. These data show that both Examples 1, 2, 4, 5, 6, 7, and 8 inhibit the transcription of cMyc in HCT116 cells.

### Selectivity Profiling Experiment: DiscoverX ScanMax

The purpose of the study is to generate an in vitro selectivity profile of the compounds of Example 8. To address selectivity, the compound of Example 8 is tested in a panel of 468 human kinases at DiscoverX Corporation using the KINOMEscan™ screening platform. KINOMEscan™ employs a novel and proprietary active site-directed competition binding assay to quantitatively measure interactions between test compounds and more than 450 human kinases and disease relevant mutant variants. KINOMEscan™ assays do not require ATP and thereby report true thermodynamic interaction affinities, as opposed to IC₅₀ values, which can depend on the ATP concentration.

Compounds that bind the kinase active site and directly (sterically) or indirectly (allosterically) prevent kinase binding to an immobilized ligand, will reduce the amount of kinase captured on the solid support. However, molecules that do not bind the kinase have no effect on the amount of kinase captured on the solid support. Compound activity is monitored by measuring the amount of kinase captured in test versus control samples by using a qPCR method that detects the associated DNA label. Further information regarding the DiscoverX Corporation KINOMEscan™ screening platform can be found at http://www.discoverx.com.

Assays to monitor binding to a 468 kinase panel were conducted at DiscoverXⓇ Corporation (Fremont, CA). Example 8 is tested at 20 µM, 2 µM, and 0.2 µM final concentrations. Kinases are tagged with DNA for qPCR detection. Streptavidin-coated magnetic beads are treated with biotinylated small molecule ligands for 30 minutes at room temperature to generate affinity resins for kinase assays. The liganded beads are blocked with excess biotin and washed with blocking buffer (SeaBlock (Pierce), 1 % BSA, 0.05 % Tween 20, 1 mM DTT) to remove unbound ligand and to reduce nonspecific binding. Binding reactions are assembled by combining kinases, liganded affinity beads, and test compounds in 1x binding buffer (20 % SeaBlock, 0.17x PBS, 0.05 % Tween 20, 6 mM DTT). Test compounds are prepared as 40x stocks in 100% DMSO and directly diluted into the assay. All reactions are performed in polypropylene 384-well plates in a final volume of 0.02 ml. The assay plates are incubated at room temperature with shaking for 1 hour and the affinity beads are washed with wash buffer (1x PBS, 0.05 % Tween 20). The beads are then re-suspended in elution buffer (1x PBS, 0.05 % Tween 20, 0.5 µM non-biotinylated affinity ligand) and incubated at room temperature with shaking for 30 minutes. The kinase concentration in the eluates is measured by qPCR. The results for primary screen binding interactions are reported as ‘% Ctrl’, where lower numbers indicate stronger hits in the matrix.

%Ctrl Calculation:

$$\left(\frac{\text{test compound signal - positive control signal}}{\text{negative control signal - positive control signal}}\right) \times 100$$

negative control = DMSO (100%Ctrl)
positive control = control compound (0%Ctrl)

The compound of Example 8 showed excellent selectivity against the 468 protein kinases panel. CDK7 was the only kinase showing less than 35% control activity at 0.2 µM concentration of the compound of Example 8. Specifically, the compound of Example 8 showed approximately 4% control activity (i.e., about 96% inhibition) against CDK7.

### Cell Proliferation Assay

The data in Table 1 shows that the compound of Example 1 inhibits proliferation and viability of the specified tumor cells lines. Cell lines are plated at the density 5000 cells per well in 100 µL per well growth medium into a white 96-well cell culture plate. See Table 1 for cell line and culture medium information. Plates are incubated at 37° C. and 5% CO₂. The following day, a serial dilution of the test compound is prepared by diluting the compound 1:3 in DMSO for 10 points. The DMSO plate is 1000X the final concentration. In addition to the CDK7 inhibitor, a DMSO alone column is included as a maximum growth control and 10 µM staurosporine final column is included as a maximum growth inhibition control. A 10X dilution plate is then prepared by adding 2 µL per well from the 1000X DMSO plate to 198 µL per well of OMEM (Life Technologies, Carlsbad, CA, cat#31985-070). Cells are treated with indicated compound by adding 11 µL per well from the 10X OMEM plate to the cell plate containing 100 µL per well growth medium for a 1X final concentration. Plates are placed back into the incubator at 37° C. and 5% CO₂. Six or seven days after compound addition, for HCC1806 or A2780 cells respectively, plates are removed from the incubator and allowed to equilibrate to RT. CELL TITER GLOⓇ reagent is thawed at room temperature and then prepared by mixing one vial of assay buffer with one vial of substrate and swirl gently to mix. CELL TITER GLOⓇ reagent is then added to the cell plate, 100 µL per well, and placed on a Titer Plate Shaker at speed setting 2 for 15 minutes at room temperature. After 15 minute incubation on shaker, luminescence is read, 1 second per well, using a Wallac VICTOR2™. Nonlinear regression and sigmoidal dose-response curves are used to calculate the half maximal inhibitory concentration (IC₅₀) with Graphpad Prism 6 software.

TABLE 1

Inhibition of cancer cell lines by the compound of Example 1

| Cell Line | Histology | IC₅₀ (µM) | Catalog Number | Media Information |
|---|---|---|---|---|
| HCC1806 | Breast Cancer | 0.01948 | ATCC# CRL-2335 | RPMI 1640 with HEPES & L-Glutamine (Gibco 22400)+ 10% FBS (Gibco cat#10082) |
| A2780 | Ovarian Cancer | 0.0228 | ATCC# CRL-2772 | RPMI 1640 with L-Glutamine (Gibco 22400) + 1X NEAA (Corning 25-025-ci) + 10% FBS (Hyclone SH30071.03) |

These data show that the compound of Example I inhibits the in vitro growth of cancer cell lines from a variety of histologies including breast and ovary, in a dose dependent manner.

### Xenograft Tumor Model

The purpose of this assay is to measure reduction in tumor volume in response to the compound of Example 1. To evaluate in vivo efficacy of a test compound, multiple xenograft tumor models are utilized. Briefly, 2.5 × 10⁶ tumor cells in a 1:1 MATRIGEL® mix (0.2 mL total volume) are injected subcutaneously into the female athymic nude mice (Envigo, Harlan Laboratories). After allowing tumors to reach a desired size of ~300-500 mm³, animals are randomized into groups of 5 for efficacy studies. Test compound is administered via oral gavage (PO) at indicated doses and regimens. Tumor growth and body weight are monitored over time to evaluate efficacy and signs of toxicity.

Test compound is formulated in 1% hydroxyethylcellulose, 0.25% polysorbate 80, 0.05% antifoam in purified water (HEC) and administered by oral gavage (final volume 0.2 mL) at the doses indicated in Table 2. A test compound is formulated on a weekly basis and stored at 4° C. Vehicles are administered to the control groups according the schedules used above using a volume of 0.2 mL per dose. Mice are dosed via oral gavage and tumor samples are collected at termination and stored at -80° C.

Tumor size and body weight are recorded and analyzed bi-weekly.

The compound of Example 1 demonstrates significant anti-tumor activity in a human cancer xenograft model (Table 2).

TABLE 2

Summary of the compound of Example 1 in-vivo single-agent efficacy (ΔT/C) in HCC1806 xenograft tumor model tested at different dose levels as indicated

| Model | Histology | Mutations | Example 1 Dose (mg/kg) | Schedule | Avg. ΔT/C |
|---|---|---|---|---|---|
| HCC1806 | Breast | KMT2C | 10 | QDx28 | 16.5 |
| HCC1806 | Breast | KMT2C | 20 | QDx28 | -38.0 |

Delta T/C% is calculated when the endpoint tumor volume in a treated group is at or above baseline tumor volume. The formula is 100×(T-T₀)/(C-C₀). Here, T and C are mean endpoint tumor volumes in the treated or control group, respectively. T₀ and C₀ are mean baseline tumor volumes in those groups.

While only certain representative compounds, materials, and method steps disclosed herein are specifically described, other combinations of the compounds, materials, and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed.

1. A compound of the formula: 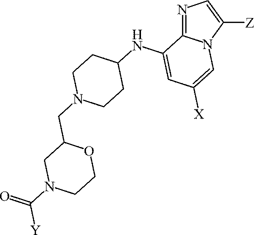
wherein,
X is —CH(OH)CH₃. —CHFCH₃. —CF₂CH₃, or —CF₃;
Y is —CH═CH₂ or—C²H═C²H₂; and
Z is —CH(CH₃)₂ or —CH(CH₃)(CH₂²H).
or a pharmaceutically acceptable salt thereof.

2. The compound according to claim 1, wherein the compound is 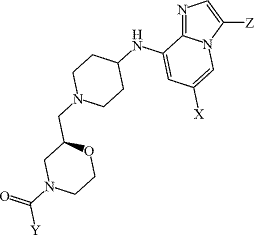
or a pharmaceutically acceptable salt thereof.

3. The compound according to claim 1, wherein the compound is 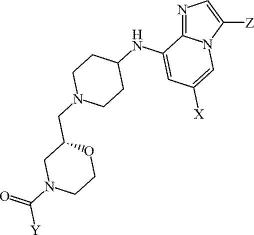
or a pharmaceutically acceptable salt thereof.

4. The compound according to claim 2, wherein the compound is 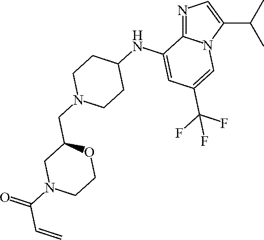 [SMILES: C=CC(=O)N1CCO[C@H](CN2CCC(Nc3cc(C(F)(F)F)cn4c(C(C)C)cnc34)CC2)C1]
or a pharmaceutically acceptable salt thereof.

5. The compound according to claim 2, wherein the compound is 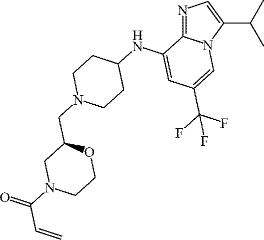 [SMILES: C=CC(=O)N1CCO[C@H](CN2CCC(Nc3cc(C(F)(F)F)cn4c(C(C)C)cnc34)CC2)C1]
.

6. The compound according to claim 2, wherein the compound is 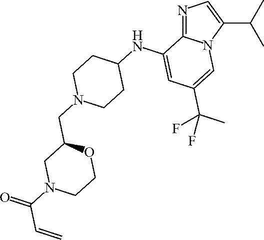 [SMILES: C=CC(=O)N1CCO[C@H](CN2CCC(Nc3cc(C(C)(F)F)cn4c(C(C)C)cnc34)CC2)C1]
or a pharmaceutically acceptable salt thereof.

7. The compound according to claim 2, wherein the compound is 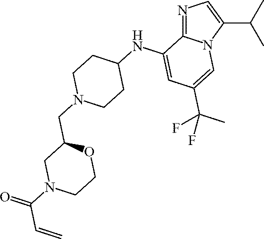 [SMILES: C=CC(=O)N1CCO[C@H](CN2CCC(Nc3cc(C(C)(F)F)cn4c(C(C)C)cnc34)CC2)C1]
.

8. The compound according to claim 1, wherein the pharmaceutically acceptable salt is a hydrochloride salt.

9. The compound according to claim 1, wherein the pharmaceutically acceptable salt is a sulfate salt.

10. A pharmaceutical composition comprising a compound of claim 1, or a pharmaceutically acceptable salt thereof, in combination with one or more pharmaceutically acceptable carriers, diluents, or excipients.

11. A method of treating urothelial cancer, uterine cancer, colorectal cancer, breast cancer, lung cancer, ovarian cancer, gastric cancer, hepatobiliary cancer, pancreatic cancer, cervical cancers, prostate cancer, hematological cancers, sarcomas, skin cancers, or gliomas, comprising administering a therapeutically effective amount of a compound of claim 1, or a pharmaceutically acceptable salt thereof, to a patient in need thereof.

12. The method according to claim 11, wherein the cancer is colorectal cancer, breast cancer, lung cancer, ovarian cancer, or gastric cancer.

13. The method according to claim 12, wherein the cancer is breast cancer.

14. The method of claim 11, wherein a biological sample from the patient contains at least one loss of function mutation in the ARID1A, KMT2C, KMT2D, or RB1 gene.

15. The method of claim 11, wherein the patient is selected for treatment if a biological sample from the patient tests positive for at least one loss of function mutation in the ARID1A. KMT2C. KMT2D, or RB1 gene.

16-22. (canceled)
